(19) United States
(12) Patent Application Publication
(10) Pub. No.: US 2018/0353512 A1
(43) Pub. Date: Dec. 13, 2018

(54) COMBINATION THERAPIES FOR TREATING B-CELL MALIGNANCIES

(71) Applicants: GILEAD SCIENCES, INC., Foster City, CA (US); ONO PHARMACEUTICAL CO., LTD., Osaka (JP)

(72) Inventors: Helen Collins, Foster City, CA (US); Julie Di Paolo, Foster City, CA (US); Kathy Keegan, Foster City, CA (US); Ryohei Kozaki, Osaka (JP); Sarah Meadows, Foster City, CA (US); Cara Nelson, Foster City, CA (US); Christophe Queva, Braine L'alleud (BE); Srinivasan Ramanathan, Foster City, CA (US); Stacey Tannheimer, Foster City, CA (US); Daniel Tumas, Foster City, CA (US); Tomoko Yasuhiro, Osaka (JP); Toshio Yoshizawa, Osaka (JP)

(21) Appl. No.: 15/739,049

(22) PCT Filed: Jun. 22, 2016
(86) PCT No.: PCT/US2016/038763

§ 371 Date: Dec. 21, 2017

Related U.S. Application Data
(60) Provisional application No. 62/183,699, filed on Jun. 23, 2015.
(60) Provisional application No. 62/200,610, filed on Aug. 3, 2015.
(60) Provisional application No. 62/263,454, filed on Dec. 4, 2015.

(51) Int. Cl. A61K 31/522; A61K 31/52; A61P 35/00

(52) U.S. Cl. CPC A61K 31/522; A61P 35/00; A61K 31/52

(57) ABSTRACT

Provided herein are methods that relate to a therapeutic strategy for treatment of a B-cell malignancy. In particular, the methods include administration of a PI3K inhibitor and a BTK inhibitor.

The present application is a national-stage entry under 35 U.S.C. § 371 of International Patent Application No. PCT/US2016/038763, filed Jun. 22, 2016, which claims priority to U.S. Provisional Application No. 62/183,699 filed Jun. 23, 2015, U.S. Provisional Application No. 62/200,610 filed Aug. 3, 2015 and U.S. Provisional Application No. 62/263,454 filed Dec. 4, 2015, the entire disclosure of each of which is incorporated by reference herein.

### FIELD

The present disclosure relates generally to therapeutics and compositions for treating B-cell malignancies, and more specifically to the use of a phosphatidylinositol 3-kinase (PI3K) inhibitor in combination with a Bruton's tyrosine kinase (BTK) inhibitor for treating B-cell malignancies.

### BACKGROUND

B-cell malignancies can arise from the accumulation of monoclonal B lymphocytes in lymph nodes and often in organs such as blood, bone marrow, spleen, and liver. This group includes histopathologic varieties such as follicular lymphoma (FL), marginal zone lymphoma (MZL), mantle cell lymphoma (MCL), chronic lymphocytic leukemia (CLL), small lymphocytic lymphoma (SLL), Waldenstrom Macroglobulinemia (WM), and diffuse large B-cell lymphoma (DLBCL). These disorders are characterized by lymphadenopathy, cytopenias, and sometimes induce life-threatening organ dysfunction. Patients may also have constitutional symptoms (fevers, night sweats, and/or weight loss) and fatigue. Few patients with B-cell malignancies are cured with available therapies. Thus, there remains a need for alternative therapies to treat B-cell malignancies in humans.

### BRIEF SUMMARY

Provided herein are methods for treating B-cell malignancies that involve the administration of a therapeutically effective amount of 2-(1-((9H-purin-6-yl)amino)propyl)-5-fluoro-3-phenylquinazolin-4(3H)-one, or a pharmaceutically acceptable salt thereof, and a therapeutically effective amount of 6-amino-9-[1-(2-butynoyl)-3-pyrrolidinyl]-7-(4-phenoxyphenyl)-7,9-dihydro-8H-purin-8-one, or a pharmaceutically acceptable salt thereof.

2-(1-((9H-Purin-6-yl)amino)propyl)-5-fluoro-3-phenylquinazolin-4(3H)-one, or a pharmaceutically acceptable salt thereof, is an example of a PI3K inhibitor. In certain variations, the 2-(1-((9H-purin-6-yl)amino)propyl)-5-fluoro-3-phenylquinazolin-4(3H)-one, or a pharmaceutically acceptable salt thereof is administered to the human at a dose between 50 mg and 150 mg.

6-Amino-9-[1-(2-butynoyl)-3-pyrrolidinyl]-7-(4-phenoxyphenyl)-7,9-dihydro-8H-purin-8-one, or a pharmaceutically acceptable salt thereof, is an example of a BTK inhibitor. In certain variations, the 6-amino-9-[1-(2-butynoyl)-3-pyrrolidinyl]-7-(4-phenoxyphenyl)-7,9-dihydro-8H-purin-8-one, or a pharmaceutically acceptable salt thereof, is administered to the human at a dose between 1 mg and 200 mg.

Provided herein are also pharmaceutical compositions, articles of manufacture and kits that comprise the PI3K inhibitor and the BTK inhibitor described herein.

### DESCRIPTION OF THE FIGURES

The present application can be understood by reference to the following description taken in conjunction with the accompanying figures.

FIG. 1A is a graph depicting cell viability in the OCI-LY10 cell line when Idelalisib was administered in combination with Compound B.

FIG. 1B is a graph depicting cell viability in the OCI-LY10 cell line when Compound B was administered in combination with Idelalisib.

FIG. 1C is a graph depicting cell viability in the TMD-8 cell line when Idelalisib was administered in combination with Compound B.

FIG. 1D is a graph depicting cell viability in the TMD-8 cell line when Compound B was administered in combination with Idelalisib.

FIG. 1E is a heat map showing cell viability in the TMD-8 cell line when Compound B was administered in combination with Idelalisib. “0”=untreated (no drug effect); “100”=completely cytostatic (no growth over assay interval); and “200”=complete cytotoxic (background signal). Further, the white line denotes clinically achievable doses.

FIG. 1F is an isobologram for the TMD-8 cell line.

FIG. 1G depicts the level of apoptosis in TMD8 cells treated with idelalisib (IDELA), Compound B (Cmpd. B), or combination of idelalisib and Compound B (IDELA+Cmpd. B).

FIG. 1H depicts graphs the cell viability of ABC DLBCL cell lines treated with Idelalisib, Compound B, and Ibrutinib.

FIGS. 2A and 2B are heat maps showing cell viability in the Rec-1 cell line (FIG. 2A) and the JVM-2 cell line (FIG. 2B) when Compound B was administered in combination with Idelalisib.

FIG. 2C is a heat map showing cell viability in the TMD-8 cell line when Compound B was administered in combination with Idelalisib.

FIG. 2D is an isobologram for the TMD-8 cell line.

FIG. 2E depicts Western Blot for phosphorylation of signaling components in cells treated with idelalisib (IDELA; 420 nM), Compound B (Cmpd. B; 320 nM) or combination of idelalisib and Compound B (IDELA+Cmpd. B), for 2 h and 24 h.

FIGS. 3A, 3B, 3C and 3D are graphs depicting growth inhibition of Ibrutinib-resistant TMD-8 with (FIGS. 3A and 3D) BTK C481F mutation, and (FIGS. 3B and 3C) A20 Q143* mutation. “TMD8^{S}” refers to the parental cell line, and “TMD8^{R}” refers to the cell line that shows resistance. The dotted line shows the effect on the TMD-8 cell line after administration of Idelalisib in combination with Compound B.

FIG. 3E shows the results of the cell viability assay of ibrutinib-resistant TMD8 clones in the presence of ibrutinib (N=4).

FIG. 4 is a graph showing TMD8 dependency on PI3Kδ for cell viability.

FIG. 5 is a graph showing acquired resistance in TMD8^{R} to idelalisib.

FIGS. 6A and 6B show PI3Kγ upregulation, and FIGS. 6C and 6D show PTEN loss.

FIG. 7 is a graph showing that TMD8^{R} were cross-resistant to Duvelisib.

FIG. 8A is an RNAseq analysis of idelalisib-sensitive and -resistant ABC-DLBCL cell lines.

FIG. 8B depicts western blots with 500 nM idelalisib for 24 h.

FIG. 8C depicts western blots that show c-Myc was inhibited with idelalisib in TMD8^{S} but not TMD8^{R}.

FIG. 8D depicts the expression of c-Myc target genes measured by RNAseq.

FIG. 9 is a graph depicting a phosphoprotein analysis.

FIGS. 10A and 10B are graphs showing that TMD8^{R} cells are cross-resistant to ibrutinib and Compound B.

FIG. 11A is a graph showing that resistance can be overcome with a combination of MK-2206 and idelalisib.

FIG. 11B is a graph showing caspase 3/7 cleavage measured at 24 h; and FIG. 11C is a graph showing Annexin measured at 48 h. Two-tailed t-test was used to calculate p-values. PI=propidium iodide.

FIG. 11D shows the results from cell viability assay of TMD8^{S} and TMD8^{R} cells treated with idelalisib, MK-2206 or combination of idelalisib and MK-2206 (1 μM) at 96 h (N=4).

FIG. 12 is a western blot showing PI3K pathway inhibition with a combination of MK-2206 and idelalisib.

FIG. 13A is a graph showing that resistance can be overcome with a combination of GSK-2334470 and idelalisib.

FIG. 13B is a graph showing caspase 3/7 cleavage measured at 24 h; and FIG. 13C is a graph showing Annexin V measured at 48 h. Two-tailed t-test was used to calculate p-values. PI=propidium iodide.

FIG. 13D shows the results from cell viability assay of TMD8^{S} and TMD8^{R} cells treated with idelalisib, GSK-2334470 or combination of idelalisib and GSK-2334470 (3 μM) at 96 h (N=4).

FIG. 14 is a western blot showing PI3K pathway inhibition with a combination of GSK-2334470 and idelalisib.

FIG. 15 is a graph showing sensitivity of FSCCL to PI3Kδ inhibition.

FIG. 16 is a graph showing less sensitivity of FSCCL^{S} and FSCCL^{R} to ibrutinib.

FIGS. 17A and 17B are graphs showing restored sensitivity in FSCCL^{R} PI3KCA mutant (N345K) to the combination of idelalisib and BYL-719.

FIG. 18A is a western blot showing the reduction of pAKT (Ser473) expression in FSCCL^{R} from the combination of idelalisib and BYL-719.

FIG. 18B is a western blot showing reduction of pAKT (Ser473) expression in IgM-stimulated FSCCL^{R} from the combination of idelalisib and BYL-719.

FIGS. 19A and 19B are western blots showing compensatory pathway activation of SPK and pSyk.

FIGS. 20A and 20B are graphs showing increased sensitivity of FSCCL^{R} SFK^{HIGH} to the combination of idelalisib and dasatinib.

FIGS. 21A and 21B are graphs showing increased sensitivity of FSCCL^{R} SFK^{HIGH} to the combination of idelalisib and entospletinib.

FIG. 22A is a RNAseq heatmap of Wnt/β-catenin signaling pathway for FSCCL^{R} clones; 4D4D6 and 2C4D9 shown as compared with FSCCL^{S}.

FIG. 22B is a western blot of untreated FSCCL^{S} and Wnt-signature FSCCL^{R} clones.

FIG. 23A depicts the results from cell viability assay in idelalisib-resistant TMD8^{R} and TMD8^{S} cells treated with idelalisib, Compound B or Compound B in combination with idelalisib.

FIG. 23B depicts the results of p-AKT S473, p-BTK Y233, c-MYC and actin in TMD8^{R} cells treated with idelalisib (IDELA, 420 nM), Compound B (Cmpd. B, 320 nM) or in combination (IDELA+Cmpd. B).

FIG. 24A shows the changes in tumor volume in the mice treated with a combination of a PI3Kδ inhibitor and a BTK inhibitor (Compound B; Cmpd. B), vehicle control, or single agent; tumor volumes are expressed as mean±SEM with p<0.05, p<0.0001 as compared to vehicle animals.

FIG. 24B show the results from Western Blot for BTK and PI3K activation in TDM8 xenograft model mice treated with a combination of a PI3Kδ inhibitor and a BTK inhibitor (Compound B; Cmpd. B), compared to vehicle control and single agent treatment; tumors from mice treated with vehicle, the PI3Kδ inhibitor (5 mg/kg), Compound B (10 mg/kg) or the PI3Kδ inhibitor+Compound B (5 mg/kg+10 mg/kg) were collected, ground and lysed. FIGS. 24C and 24D show the quantitation of averages of the tumors from mice in each treatment group; proteins were quantitated by AUC, p-BTK Y223 was normalized to total BTK protein, p-S6RP S235/236 was normalized to actin, mean±SD.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein is a method for treating B-cell malignancy in a human in need thereof, comprising administering a therapeutically effective amount of 2-(1-((9H-purin-6-yl)amino)propyl)-5-fluoro-3-phenylquinazolin-4(3H)-one, or a pharmaceutically acceptable salt thereof, and a therapeutically effective amount of 6-amino-9-[1-(2-butynoyl)-3-pyrrolidinyl]-7-(4-phenoxyphenyl)-7,9-dihydro-8H-purin-8-one, or a pharmaceutically acceptable salt thereof. Provided are also compositions (including pharmaceutical compositions, formulations, or unit dosages), articles of manufacture and kits comprising the PI3K inhibitor and the BTK inhibitor described herein. Also provided is the use of a compound of 2-(1-((9H-purin-6-yl)amino)propyl)-5-fluoro-3-phenylquinazolin-4(3H)-one and 6-amino-9-[1-(2-butynoyl)-3-pyrrolidinyl]-7-(4-phenoxyphenyl)-7,9-dihydro-8H-purin-8-one, or a pharmaceutically acceptable salt thereof, in the manufacture of a medicament for treating B-cell malignancy. Additionally provided is the use of a compound of 2-(1-((9H-purin-6-yl)amino)propyl)-5-fluoro-3-phenylquinazolin-4(3H)-one or a pharmaceutically acceptable salt thereof, and 6-amino-9-[1-(2-butynoyl)-3-pyrrolidinyl]-7-(4-phenoxyphenyl)-7,9-dihydro-8H-purin-8-one, or a pharmaceutically acceptable salt thereof, in the manufacture of a medicament for treating B-cell malignancy. Also provided is the use of 2-(1-((9H-purin-6-yl)amino)propyl)-5-fluoro-3-phenylquinazolin-4(3H)-one and 6-amino-9-[1-(2-butynoyl)-3-pyrrolidinyl]-7-(4-phenoxyphenyl)-7,9-dihydro-8H-purin-8-one, or a pharmaceutically acceptable salt thereof, for treating B-cell malignancy.

### Compounds

2-(1-((9H-Purin-6-yl)amino)propyl)-5-fluoro-3-phenylquinazolin-4(3H)-one, or a pharmaceutically acceptable salt thereof, is an example of a PI3K inhibitor, and more specifically, a PI3 kinase delta-specific isoform (PI3Kδ) inhibitor. Such compound is also referred to in the art as Idelalisib, and referred to herein as Compound A, and has the structure:

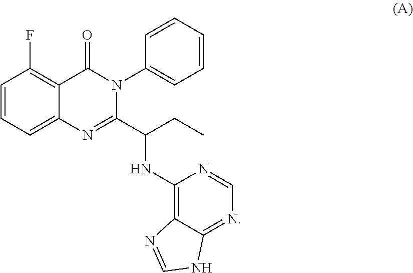 [SMILES: CCC(Nc1ncnc2[nH]cnc12)c1nc2cccc(F)c2c(=O)n1-c1ccccc1]

In one variation, Compound A is predominantly the S-enantiomer, having the structure:

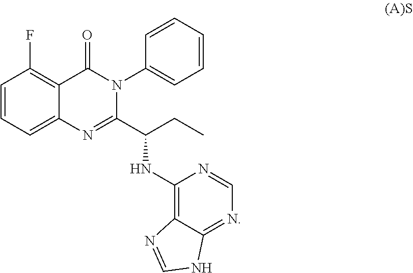 [SMILES: CC[C@H](Nc1ncnc2[nH]cnc12)c1nc2cccc(F)c2c(=O)n1-c1ccccc1]

The (S)-enantiomer of Compound A may also be referred to by its compound name: (S)-2-(1-((9H-purin-6-yl)amino)propyl)-5-fluoro-3-phenylquinazolin-4(3H)-one.

Compound A may be synthesized according to the methods described in U.S. Pat. No. 7,932,260.

6-Amino-9-[1-(2-butynoyl)-3-pyrrolidinyl]-7-(4-phenoxyphenyl)-7,9-dihydro-8H-purin-8-one, or a pharmaceutically acceptable salt thereof, is an example of a BTK inhibitor. Such compound is also referred to herein as Compound B, and has the structure:

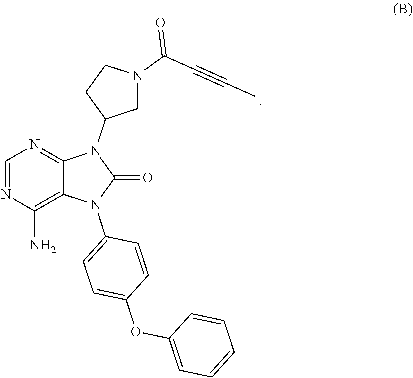 [SMILES: CC#CC(=O)N1CCC(n2c(=O)n(-c3ccc(Oc4ccccc4)cc3)c3c(N)ncnc32)C1]

In one variation, Compound B is predominantly the (R)-enantiomer, having the structure:

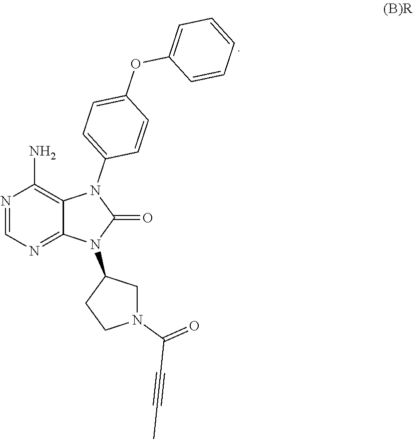 [SMILES: CC#CC(=O)N1CC[C@@H](n2c(=O)n(-c3ccc(Oc4ccccc4)cc3)c3c(N)ncnc32)C1]

The (R)-enantiomer of Compound B may also be referred to by its compound name: 6-amino-9-[(3R)-1-(2-butynoyl)-3-pyrrolidinyl]-7-(4-phenoxyphenyl)-7,9-dihydro-8H-purin-8-one.

In some embodiments, the BTK inhibitor is a salt of Compound B. For example, in some variations, the BTK inhibitor is a hydrochloride salt of Compound B. In one variation, the BTK inhibitor is a monohydrochloride salt of Compound B.

Compound B may be synthesized according to the methods described in U.S. Pat. No. 8,557,803.

The compound names provided herein are named using ChemBioDraw Ultra 14.0. One skilled in the art understands that the compound may be named or identified using various commonly recognized nomenclature systems and symbols. By way of example, the compound may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

Also provided herein are isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g. humans). Also provided for isotopically labeled compounds described herein are any pharmaceutically acceptable salts, or hydrates, as the case may be.

In some variations, the compounds disclosed herein may be varied such that from 1 to n hydrogens attached to a carbon atom is/are replaced by deuterium, in which n is the number of hydrogens in the molecule. Such compounds may exhibit increased resistance to metabolism and are thus useful for increasing the half-life of the compound when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The term “pharmaceutically acceptable” with respect to a substance refers to that substance which is generally regarded as safe and suitable for use without undue toxicity, irritation, allergic response, and the like, commensurate with a reasonable benefit/risk ratio.

“Pharmaceutically acceptable salt” refers to a salt of a compound (e.g., of Compound A or Compound B, or both) that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

### Methods of Treatment

The PI3K and BTK inhibitors described herein may be used in a combination therapy. Accordingly, provided herein is a method for treating B-cell malignancy in a human in need thereof, comprising administering to the human a therapeutically effective amount of the PI3K inhibitor and a therapeutically effective amount of the BTK inhibitor, as described herein.

In some variations, “treatment” or “treating” is an approach for obtaining beneficial or desired results including clinical results. Beneficial or desired clinical results may include one or more of the following:

(i) inhibiting the disease or condition (e.g., decreasing one or more symptoms resulting from the disease or condition, and/or diminishing the extent of the disease or condition);

(ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or

(iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

In some variations, “delaying” the development of a disease or condition means to defer, hinder, slow, retard, stabilize, and/or postpone development of the disease or condition. This delay can be of varying lengths of time, depending on the history of the disease or condition, and/or subject being treated. For example, a method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

In some embodiments, the administration of the PI3K inhibitor and the BTK inhibitor described herein may unexpectedly reduce side effects associated with the administration of the PI3K inhibitor alone or the BTK inhibitor alone. For example, in one variation, the reduction in side effects may be a reduction in the frequency of the side effects. In some embodiments, the administration of the PI3K inhibitor and the BTK inhibitor reduces the frequency of diarrhea, colitis, transaminase elevation, rash, or pneumonitis, or any combinations thereof. In another variation, the reduction in side effects may be a reduction in the severity of the side effects. In some embodiments, the administration of the PI3K inhibitor and the BTK inhibitor reduces the severity of diarrhea, colitis, transaminase elevation, rash, or pneumonitis, or any combinations thereof. In other embodiments, the administration of the PI3K inhibitor and the BTK inhibitor described herein may unexpectedly result in little or no increase in side effects associated with the administration of the PI3K inhibitor alone or the BTK inhibitor alone. In other embodiments, the administration of the PI3K inhibitor and the BTK inhibitor results in little or no increase in diarrhea, colitis, transaminase elevation, rash, or pneumonitis, or any combinations thereof.

The administration of the PI3K inhibitor and the BTK inhibitor described herein may unexpectedly reverse, or at least partially reverse, resistance to a BTK therapy, a PI3K therapy, or a combination thereof. In some aspects, provided herein are methods for treating a human resistant to a BTK inhibitor alone, a PI3K inhibitor alone, or a combination thereof, comprising administering to the human a therapeutically effective amount of the PI3K inhibitor and a therapeutically effective amount of the BTK inhibitor, as described herein.

In some aspects, inhibition of both PI3K and BTK signaling pathways may act synergistically to overcome resistance to PI3K or BTK inhibitors. In some aspects, inhibition of both pathways may suppress PI3K, BTK and/or MAPK pathways in an additive or synergistic manner. The synergistic response may result in the reduced dosage of PI3K and/or BTK inhibitors, shorten the treatment time, or increase patient response to treatment.

In some embodiments, the human having resistance to therapy comprising a BTK inhibitor alone and/or a PI3K inhibitor alone may have a tumor necrosis factor α-induced protein 3 (TNFAIP3, also known as A20) mutation. In yet other aspects, provided is a method for treating a B-cell malignancy in a human, comprising: a) selecting a human having a tumor necrosis factor α-induced protein 3 (TNFAIP3, also known as A20) mutation; and b) administering to the human a therapeutically effective amount of the PI3K inhibitor and a therapeutically effective amount of the BTK inhibitor, as described herein. In certain embodiments, the human having resistance to therapy comprising a BTK inhibitor alone and/or a PI3K inhibitor alone may have BTK C481 mutation. In certain other aspects, provided is a method for treating a B-cell malignancy in a human, comprising: a) selecting a human having BTK C481F mutation; and b) administering to the human a therapeutically effective amount of the PI3K inhibitor and a therapeutically effective amount of the BTK inhibitor as described herein.

In one variation, provided herein are methods for treating a human resistant to a BTK inhibitor alone, comprising administering to the human a therapeutically effective amount of the PI3K inhibitor and a therapeutically effective amount of the BTK inhibitor, as described herein. In other variations, provided herein are methods for treating a human resistant to a PI3K inhibitor alone, comprising administering to the human a therapeutically effective amount of the PI3K inhibitor and a therapeutically effective amount of the BTK inhibitor as described herein.

B-Cell Malignancies

In some embodiments, the B-cell malignancy is a B-cell lymphoma or a B-cell leukemia. In some variations, the B-cell malignancy is follicular lymphoma (FL), marginal zone lymphoma (MZL), small lymphocytic lymphoma (SLL), chronic lymphocytic leukemia (CLL), mantle cell lymphoma (MCL), Waldenstrom Macroglobulinemia (WM), non-germinal center B-cell lymphoma (GCB), or diffuse large B-cell lymphoma (DLBCL).

In some variations, the B-cell malignancy is diffuse large B-cell lymphoma (DLBCL). In one variation, the DLBCL is activated B-cell like diffuse large B-cell lymphoma (ABC-DLBCL). In another variation, the DLBCL is germinal center B-cell like diffuse large B-cell lymphoma (GCB-DLBCL). In other variations, the DLBCL is a non-GCB DLBCL.

In other variations, the B-cell malignancy is chronic lymphocytic leukemia (CLL). In other variations, the B-cell malignancy is mantle cell lymphoma (MCL). In yet other variations, the B-cell malignancy is Waldenstrom Macroglobulinemia (WM).

In some variations, the B-cell malignancy is indolent non-Hodgkin's lymphoma.

Subject

The human in need thereof may be an individual who has or is suspected of having a B-cell malignancy. In some of variations, the human is at risk of developing a B-cell malignancy (e.g., a human who is genetically or otherwise predisposed to developing a B-cell malignancy) and who has or has not been diagnosed with the B-cell malignancy. As used herein, an “at risk” subject is a subject who is at risk of developing B-cell malignancy. The subject may or may not have detectable disease, and may or may not have displayed detectable disease prior to the treatment methods described herein. An at risk subject may have one or more so-called risk factors, which are measurable parameters that correlate with development of a B-cell malignancy, such as described herein. A subject having one or more of these risk factors has a higher probability of developing a B-cell malignancy than an individual without these risk factor(s).

These risk factors may include, for example, age, sex, race, diet, history of previous disease, presence of precursor disease, genetic (e.g., hereditary) considerations, and environmental exposure. In some embodiments, a human at risk for a B-cell malignancy includes, for example, a human whose relatives have experienced this disease, and those whose risk is determined by analysis of genetic or biochemical markers. Prior history of having a B-cell malignancy may also be a risk factor for instances of B-cell malignancy recurrence.

In some embodiments, provided herein is a method for treating a human who exhibits one or more symptoms associated with a B-cell malignancy. In some embodiments, the human is at an early stage of a B-cell malignancy. In other embodiments, the human is at an advanced stage of a B-cell malignancy.

In some embodiments, provided herein is a method for treating a human who is undergoing one or more standard therapies for treating a B-cell malignancy, such as chemotherapy, radiotherapy, immunotherapy, and/or surgery. Thus, in some foregoing embodiments, the combination of a PI3K inhibitor and a BTK inhibitor, as described herein, may be administered before, during, or after administration of chemotherapy, radiotherapy, immunotherapy, and/or surgery.

In another aspect, provided herein is a method for treating a human who is “refractory” to a B-cell malignancy treatment or who is in “relapse” after treatment for a B-cell malignancy. A subject “refractory” to an anti-B-cell malignancy therapy means they do not respond to the particular treatment, also referred to as resistant. The B-cell malignancy may be resistant to treatment from the beginning of treatment, or may become resistant during the course of treatment, for example after the treatment has shown some effect on the B-cell malignancy, but not enough to be considered a remission or partial remission. A subject in “relapse” means that the B-cell malignancy has returned or the signs and symptoms of the B-cell malignancy have returned after a period of improvement, e.g. after a treatment has shown effective reduction in the B-cell malignancy, such as after a subject is in remission or partial remission.

In some variations, the human is (i) refractory to at least one anti-B-cell malignancy therapy, or (ii) in relapse after treatment with at least one anti-B-cell malignancy therapy, or both (i) and (ii). In some of embodiments, the human is refractory to at least two, at least three, or at least four anti-B-cell malignancy therapies (including, for example, standard or experimental chemotherapies). In one variation, the human is (i) refractory to a BTK therapy, a PI3K therapy, or a combination thereof; or (ii) in relapse after treatment with a BTK therapy, a PI3K therapy, or a combination thereof; or both (i) and (ii). In additional variation, the human is (i) refractory to a BTK therapy or a combination thereof; or (ii) in relapse after treatment with a BTK therapy or a combination thereof; or both (i) and (ii). In some additional variation, the human is (i) refractory to a PI3K therapy or a combination thereof; or (ii) in relapse after treatment with a PI3K therapy or a combination thereof; or both (i) and (ii). In other variation, the human is refractory to a BTK therapy; or (ii) in relapse after treatment with a BTK therapy; or both (i) and (ii). In certain other variation, the human is (i) refractory a PI3K therapy or (ii) in relapse after treatment with a PI3K therapy; or both (i) and (ii).

In certain variations, the human is (i) refractory to at least one chronic lymphocytic leukemia therapy, or (ii) in relapse after treatment with at least one chronic lymphocytic leukemia therapy, or both (i) and (ii). In one variation, the chronic lymphocytic leukemia therapies that a human may have received include, for example, regimens of:
a) fludarabine (Fludara®);
b) rituximab (Rituxan®);
c) rituximab (Rituxan®) combined with fludarabine (sometimes abbreviated as FR);
d) cyclophosphamide (Cytoxan®) combined with fludarabine; cyclophosphamide combined with rituximab and fludarabine (sometimes abbreviated as FCR);
e) cyclophosphamide combined with vincristine and prednisone (sometimes abbreviated as CVP);
f) cyclophosphamide combined with vincristine, prednisone, and rituximab;
g) combination of cyclophosphamide, doxorubicin, vincristine (Oncovin), and prednisone (sometimes referred to as CHOP);
h) Chlorambucil combined with prednisone, rituximab, obinutuzumab, or ofatumumab
i) pentostatin combined with cyclophosphamide and rituximab (sometimes abbreviated as PCR);
j) bendamustine (Treanda®) combined with rituximab ((sometimes abbreviated as BR);
k) alemtuzumab (Campath®);
l) fludarabine plus cyclophosphamide, bendamustine, or chlorambucil; and
m) fludarabine plus cyclophosphamide, bendamustine, or chlorambucil, combined with an anti-CD20 antibody, such as rituximab, ofatumumab, or obinutuzumab.

In another aspect, provided is a method for sensitizing a human who is (i) refractory to at least one chemotherapy treatment, or (ii) in relapse after treatment with chemotherapy, or both (i) and (ii), wherein the method comprises administering a PI3K inhibitor in combination with a BTK inhibitor, as described herein, to the human. A human who is sensitized is a human who is responsive to the treatment involving administration of a PI3K inhibitor in combination with a BTK inhibitor, as described herein, or who has not developed resistance to such treatment. In one variation, the human is (i) refractory to a BTK therapy, a PI3K therapy, or a combination thereof; or (ii) in relapse after treatment with a BTK therapy, a PI3K therapy, or a combination thereof; or both (i) and (ii).

In yet another aspect, provided is a method for treating a human resistant to a BTK therapy, a PI3K therapy, or a combination thereof, comprising administering a PI3K inhibitor in combination with a BTK inhibitor, as described herein, to the human. In some embodiments, the administration of the PI3K inhibitor in combination with the BTK inhibitor increases cell apoptosis by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to cell apoptosis in the human when a BTK therapy or a PI3K therapy is administered to the human. In one variation, the administration of the PI3K inhibitor in combination with the BTK inhibitor increases cell apoptosis by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to cell apoptosis in the human when a therapy comprising a BTK inhibitor as the only active agent is administered to the human. In another variation, the administration of the PI3K inhibitor in combination with the BTK inhibitor increases cell apoptosis by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to cell apoptosis in the human when a therapy comprising a PI3K inhibitor as the only active agent is administered to the human.

In some embodiments, the human having resistance to a BTK therapy, a PI3K therapy, or a combination thereof, may have a tumor necrosis factor α-induced protein 3 (TNFAIP3, also known as A20) mutation. In yet other aspects, provided is a method for treating a B-cell malignancy in a human, comprising: a) selecting a human having a tumor necrosis factor α-induced protein 3 (TNFAIP3, also known as A20) mutation; and b) administering to the human a therapeutically effective amount of the PI3K inhibitor and a therapeutically effective amount of the BTK inhibitor, as described herein.

In some variations, a BTK therapy is a therapy where the only active agent is a BTK inhibitor. By way of example, BTK inhibitor includes and is not limited to Compound B, ibrutinib (which may also be referred to as 1-[(3R)-3-[4-Amino-3-(4-phenoxyphenyl)-1H-pyrazolo[3,4-d]pyrimidin-1-yl]piperidin-1-yl]prop-2-en-1-one), and acalabrutinib (which may be referred to as 4-{8-Amino-3-[(2S)-1-(2-butynoyl)-2-pyrrolidinyl]imidazo[1,5-a]pyrazin-1-yl}-N-(2-pyridinyl)benzamide). In some variations, a PI3K therapy is a therapy where the only active agent is a PI3K inhibitor. By way of example, PI3K inhibitor includes and is not limited to Compound A (which may also be referred to as Idelalisib, idelalisib, or IDELA, or 2-(1-((9H-Purin-6-yl)amino)propyl)-5-fluoro-3-phenylquinazolin-4(3H)-one), duvelisib (which may also be referred to as 8-Chloro-2-phenyl-3-[(1S)-1-(3H-purin-6-ylamino)ethyl]-1(2H)-isoquinolinone), TGR1202, and alpelisib (which may also be referred to as BYL719).

In another aspect, provided herein are methods for treating a human for a B-cell malignancy, with comorbidity, wherein the treatment is also effective in treating the comorbidity. A “comorbidity” to B-cell malignancy is a disease that occurs at the same time as the B-cell malignancy.

In other aspects, provided herein are methods for treating cancer in a human in need thereof, comprising administering to the human a therapeutically effective amount of Compound A, or a pharmaceutically acceptable salt thereof, and a therapeutically effective amount of Compound B, or a pharmaceutically acceptable salt thereof. In some embodiments, the cancer is pancreatic cancer, urological cancer, bladder cancer, colorectal cancer, colon cancer, breast cancer, prostate cancer, renal cancer, hepatocellular cancer, thyroid cancer, gall bladder cancer, lung cancer (e.g. non-small cell lung cancer, small-cell lung cancer), ovarian cancer, cervical cancer, gastric cancer, endometrial cancer, esophageal cancer, head and neck cancer, melanoma, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, esophageal squamous cell carcinomas, oral carcinomas, cancers of the adrenal cortex, or ACTH-producing tumors. In one variation, the cancer is pancreatic cancer.

Therapeutically Effective Amounts

In some variations, a therapeutically effective amount refers to an amount that is sufficient to effect treatment, as defined below, when administered to a subject (e.g., a human) in need of such treatment. The therapeutically effective amount will vary depending upon the subject and disease condition being treated, the weight and age of the subject, the severity of the disease condition, the manner of administration and the like, which can readily be determined by one of ordinary skill in the art. For example, in one variation, a therapeutically effective amount of Compound A, or a pharmaceutically acceptable salt thereof, is an amount sufficient to modulate PI3K expression, and thereby treat a human suffering an indication, or to ameliorate or alleviate the existing symptoms of the indication. In one variation, a therapeutically effective amount of Compound B, or a pharmaceutically acceptable salt thereof, is an amount sufficient to modulate BTK activity, and thereby treat a human suffering an indication, or to ameliorate or alleviate the existing symptoms of the indication.

In another variation, the therapeutically effective amount of the PI3K inhibitor, such as Compound A, or a pharmaceutically acceptable salt thereof, may be an amount sufficient to decrease a symptom of a disease or condition responsive to inhibition of PI3K activity. In another variation, the therapeutically effective amount of the BTK inhibitor, such as Compound B, or a pharmaceutically acceptable salt thereof, may be an amount sufficient to decrease BTK activity.

In certain variations, the administration to the human in need thereof of the therapeutically effective amounts of the PI3K inhibitor and the BTK inhibitor:

(i) reduces the frequency and/or severity of at least one adverse event when administered to the human; or

(ii) has little or no increase in the frequency and/or severity of at least one adverse event when administered to the human; or

a combination of (i) and (ii).

In some variations, the adverse events may include diarrhea, colitis, transaminase elevation, rash, and pneumonitis.

In some variations, the PI3K inhibitor, such as Compound A, or a pharmaceutically acceptable salt thereof, is administered to the human at a dose not more than 150 mg, or less than 150 mg; or between 40 mg and 150 mg, between 50 mg and 150 mg, between 50 mg and 100 mg, or between 50 mg and 75 mg; or about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

For example, in one variation, the PI3K inhibitor, such as Compound A, or a pharmaceutically acceptable salt thereof, is administered at a dose less than 150 mg, and when administered at such dose in combination with the BTK inhibitor, (i) reduces and/or (ii) has little to no increase in the frequency and/or severity of at least one adverse event when a combination of the PI3K and the BTK inhibitors are administered to the human. In certain variations, the administration of a combination of the PI3K and the BTK inhibitors is at least as effective in treating the B-cell malignancy (e.g., anti-proliferative activity, progression free survival, overall response rate) as compared to administration of 150 mg of the PI3K inhibitor, such as Compound A, or a pharmaceutically acceptable salt thereof, alone.

In another variation, the PI3K inhibitor, such as Compound A, or a pharmaceutically acceptable salt thereof, is administered at a dose not more than 150 mg, and when administered at such dose in combination with the BTK inhibitor, (i) reduces and/or (ii) has little to no increase in the frequency and/or severity of at least one adverse event when a combination of the PI3K and the BTK inhibitors are administered to the human. In certain variations, the administration of a combination of the PI3K and the BTK inhibitors is at least as effective in treating the B-cell malignancy (including, for example, inducing anti-proliferative activity in the human) as compared to administration of 150 mg of the PI3K inhibitor, such as Compound A, or a pharmaceutically acceptable salt thereof, alone.

In some variations, the BTK inhibitor, such as Compound B, or a pharmaceutically acceptable salt thereof, is administered to the human at a dose between 1 mg to 600 mg, between 40 mg and 600 mg, between 1 mg and 250 mg, between 1 mg and 200 mg, between 1 mg and 175 mg, between 1 mg and 160 mg, between 1 mg and 100 mg, between 5 mg and 50 mg, or between 5 mg and 30 mg; or about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, or about 145 mg.

In certain variations, the BTK inhibitor, such as Compound B, or a pharmaceutically acceptable salt thereof, is administered to the human at a dose between 40 mg and 1200 mg, between 40 mg and 800 mg, between 40 mg and 600 mg, between 40 mg and 400 mg, about 40 mg, about 100 mg, about 200 mg, about 300 mg, about 400 mg, about 500 mg, about 600 mg, about 700 mg, or about 800 mg.

The therapeutically effective amount of the PI3K and BTK inhibitors may be provided in a single dose or multiple doses to achieve the desired treatment endpoint. As used herein, “dose” refers to the total amount of an active ingredient to be taken each time by a human. The dose administered, for example for oral administration described above, may be administered once daily (QD), twice daily (BID), three times daily, four times daily, or more than four times daily. In some embodiments, the PI3K and/or the BTK inhibitors may be administered once daily. In some embodiments, the PI3K and/or the BTK inhibitors may be administered twice daily. In yet other embodiments, the PI3K and/or the BTK inhibitors may be administered once weekly.

In one variation, the PI3K inhibitor, such as Compound A, or a pharmaceutically acceptable salt thereof, is administered to the human at a dose of 50 mg twice daily. In another variation, the PI3K inhibitor, such as Compound A, or a pharmaceutically acceptable salt thereof, is administered to human at a dose of 100 mg once daily.

In another variation, the BTK inhibitor, such as Compound B, or a pharmaceutically acceptable salt thereof, is administered to the human at a dose of between 40 mg and 150 mg, or about 20 mg, about 40 mg, or about 75 mg, twice daily. In yet another variation, the BTK inhibitor, such as Compound B, or a pharmaceutically acceptable salt thereof, is administered to the human at a dose of between 40 mg and 80 mg once daily.

For example, in certain variations, the PI3K inhibitor, such as Compound A, or a pharmaceutically acceptable salt thereof, is administered to the human at a dose of about 50 mg twice daily; and the BTK inhibitor, such as Compound B, or a pharmaceutically acceptable salt thereof, is administered to the human at a dose of between 20 mg ad 150 mg, or about 20 mg, or about 40 mg, or about 80 mg, or about 150 mg, once daily. In other variations, the PI3K inhibitor, such as Compound A, or a pharmaceutically acceptable salt thereof, is administered to the human at a dose of about 50 mg twice daily; and the BTK inhibitor, such as Compound B, or a pharmaceutically acceptable salt thereof, is administered to the human at a dose of between 20 mg and 75 mg, or about 20 mg, or about 40 mg, or about 75 mg, twice daily.

In certain other variations, the PI3K inhibitor, such as Compound A, or a pharmaceutically acceptable salt thereof, is administered to the human at a dose of about 100 mg twice daily; and the BTK inhibitor, such as Compound B, or a pharmaceutically acceptable salt thereof, is administered to the human at a dose of between 20 mg and 150 mg, or about 20 mg, or about 40 mg, or about 80 mg, or about 150 mg, once daily. In other variations, the PI3K inhibitor, such as Compound A, or a pharmaceutically acceptable salt thereof, is administered to the human at a dose of about 100 mg twice daily; and the BTK inhibitor, such as Compound B, or a pharmaceutically acceptable salt thereof, is administered to the human at a dose of between 20 mg and 75 mg, or about 20 mg, or about 40 mg, or about 75 mg, twice daily.

In certain variations, the PI3K inhibitor is dosed prior to dosing with the BTK inhibitor. For example, in a certain variation, the PI3K inhibitor is dosed at 50 mg to 150 mg twice daily for a specified period of time, followed by co-administration with the BTK inhibitor. In certain variations, the PI3K inhibitor is dosed for a period of up to about 12 weeks prior to co-administration with the BTK inhibitor. In certain variations, the PI3K inhibitor is dosed for a period of about 1 to 12 weeks, 4 to 12 weeks, 6 to 12 weeks, 8 to 12 weeks, 10 to 12 weeks, 2 weeks, 3 weeks, 4 weeks, 5 weeks, 6 weeks, 7 weeks, 8 weeks, 9 weeks, 10 weeks, 11 weeks or 12 weeks prior to co-administration with the BTK inhibitor. In a certain variation, the PI3K inhibitor is dosed for a period of about 4 to 12 weeks or about 6 to 12 weeks prior to co-administration with the BTK inhibitor. In certain variations, the PI3K inhibitor is dosed at 50 mg to 150 mg twice daily for a specified period of time, followed by co-administration with the BTK inhibitor, wherein the BTK inhibitor is administered at a dose between 40 mg and 1200 mg, between 40 mg and 800 mg, between 40 mg and 600 mg, between 40 mg and 400 mg, about 40 mg, about 100 mg, about 200 mg, about 300 mg, about 400 mg, about 500 mg, about 600 mg, about 700 mg, or about 800 mg.

In certain variations, the BTK inhibitor is dosed prior to dosing with the PI3K inhibitor. For example, in a certain variation, the BTK inhibitor is dosed between 40 mg and 1200 mg, between 40 mg and 800 mg, between 40 mg and 600 mg, between 40 mg and 400 mg, about 40 mg, about 100 mg, about 200 mg, about 300 mg, about 400 mg, about 500 mg, about 600 mg, about 700 mg, or about 800 mg daily or weekly for a specified period of time, followed by co-administration with the PI3K inhibitor. In certain variations, the BTK inhibitor is dosed for a period of up to about 12 weeks prior to co-administration with the PI3K inhibitor. In certain variations, the BTK inhibitor is dosed for a period of about 1 to 12 weeks, 4 to 12 weeks, 6 to 12 weeks, 8 to 12 weeks, 10 to 12 weeks, 2 weeks, 3 weeks, 4 weeks, 5 weeks, 6 weeks, 7 weeks, 8 weeks, 9 weeks, 10 weeks, 11 weeks or 12 weeks prior to co-administration with the PI3K inhibitor. In a certain variation, the BTK inhibitor is dosed for a period of about 4 to 12 weeks or about 6 to 12 weeks prior to co-administration with the PI3K inhibitor. In certain variations, the BTK inhibitor is dosed at between 40 mg and 1200 mg, between 40 mg and 800 mg, between 40 mg and 600 mg, between 40 mg and 400 mg, about 40 mg, about 100 mg, about 200 mg, about 300 mg, about 400 mg, about 500 mg, about 600 mg, about 700 mg, or about 800 mg daily or weekly for a specified period of time, followed by co-administration with the PI3K inhibitor, wherein the PI3K inhibitor is dosed from 50 mg to 150 mg twice daily.

In some variations, the therapeutically effective amount of each of the compounds, such as Compound A, or a pharmaceutically acceptable salt thereof, in combination with Compound B, or a pharmaceutically acceptable salt thereof, is reduced compared to the doses for single agent administration.

In some aspects, the combination of administration of a PI3K inhibitor, such as Compound A, and a BTK inhibitor, such as Compound B, allows administration of reduced doses of each drug, thus limiting the toxicity of each drug. In some examples, the combination allows reduced dose administration compared to single agent administration. For Example, the PI3K inhibitor, such as Compound A, and the BTK inhibitor, such as Compound B, are dosed between 1 mg and 2000 mg, between 5 mg and 2000 mg, between 10 mg and 2000 mg, between 20 mg and 2000 mg, between 30 mg and 2000 mg, between 40 mg and 2000 mg, between 40 mg and 1200 mg, between 40 mg and 800 mg, between 40 mg and 600 mg, between 40 mg and 400 mg, such as about 1 mg, about 2 mg, about 3 mg, about 4 mg, about 5 mg, about 10 mg, about 20 mg, about 30 mg, about 40 mg, about 100 mg, about 200 mg, about 300 mg, about 400 mg, about 500 mg, about 600 mg, about 700 mg, or about 800 mg daily or weekly for a specified period of time. In some aspects provided herein, each of PI3K inhibitor (such as Idelalisib) and BTK inhibitor (such as Compound B) of the combination therapy may be administered at reduced doses compared to each PI3K inhibitor or BTK inhibitor of the single therapy.

Administration

The PI3K inhibitor, such as Compound A, or a pharmaceutically acceptable salt thereof, and the BTK inhibitor, such as Compound B, or a pharmaceutically acceptable salt thereof, may be administered using any suitable methods known in the art. For example, the compounds may be administered bucally, ophthalmically, orally, osmotically, parenterally (intramuscularly, intraperitoneally intrasternally, intravenously, subcutaneously), rectally, topically, transdermally, or vaginally. In one variation, the PI3K inhibitor and the BTK inhibitor are each administered orally.

Further, in certain variations, the PI3K inhibitor, such as Compound A, or a pharmaceutically acceptable salt thereof, may be administered prior, after or concurrently with the BTK inhibitor, such as Compound B, or a pharmaceutically acceptable salt thereof, described herein. Furthermore, in some variations, the BTK inhibitor, such as Compound B, or a pharmaceutically acceptable salt thereof, may be administered prior to, after or concurrently with the PI3K inhibitor, such as Compound A, or a pharmaceutically acceptable salt thereof, described herein.

Pharmaceutical Compositions

The PI3K and BTK inhibitors may be administered in the form of pharmaceutical compositions. For example, in some variations, the PI3K inhibitor described herein may be present in a pharmaceutical composition comprising the PI3K inhibitor, and at least one pharmaceutically acceptable vehicle. In some variations, the BTK inhibitor described herein may be present in a pharmaceutical composition comprising the BTK inhibitor, and at least one pharmaceutically acceptable vehicle. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

This disclosure therefore provides pharmaceutical compositions that contain the PI3K and BTK inhibitors as described herein, and one or more pharmaceutically acceptable vehicle, such as excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. In a certain embodiment, the pharmaceutical composition is administered orally in either single or multiple doses.

In some embodiments, the pharmaceutical compositions described herein are formulated in a unit dosage form. The term “unit dosage forms” refers to physically discrete units suitable as unitary dosages for human subjects, each unit containing a predetermined quantity of active material calculated to produce the desired therapeutic effect, in association with a suitable pharmaceutical excipient. In some variations, the pharmaceutical compositions described herein are in the form of a tablet, capsule, or ampoule.

In certain embodiments, the PI3K inhibitor described herein, such as Compound A, or a pharmaceutically acceptable salt thereof, is formulated as a tablet. In certain embodiments, the BTK inhibitor described herein, such as Compound B, or a pharmaceutically acceptable salt thereof, is also formulated as a tablet. In some variations, Compound A, or a pharmaceutically acceptable salt thereof, and Compound B, or a pharmaceutically acceptable salt thereof, are formulated as separate tablets. In other variations, Compound A, or a pharmaceutically acceptable salt thereof, and Compound B, or a pharmaceutically acceptable salt thereof, are formulated as a single tablet.

Additional Therapeutic Agents

In the present disclosure, in some aspects, the combination (e.g., of the PI3K inhibitor and the BTK inhibitor) described herein may be used or combined with a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof.

Chemotherapeutic agents may be categorized by their mechanism of action into, for example, the following groups: anti-metabolites/anti-cancer agents, such as pyrimidine analogs (floxuridine, capecitabine, and cytarabine); purine analogs, folate antagonists and related inhibitors antiproliferative/antimitotic agents including natural products such as vinca alkaloid (vinblastine, vincristine) and microtubule such as taxane (paclitaxel, docetaxel), vinblastin, nocodazole, epothilones and navelbine, epidipodophyllotoxins (etoposide, teniposide); DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide, triethylenethiophosphoramide); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prednisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin.

As used herein the term “chemotherapeutic agent” or “chemotherapeutic” (or “chemotherapy,” in the case of treatment with a chemotherapeutic agent) is meant to encompass any non-proteinaceous (i.e, non-peptidic) chemical compound useful in the treatment of cancer. Examples of chemotherapeutic agents include alkylating agents such as thiotepa and cyclophosphamide (CYTOXAN®); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammaII and calicheamicin phiII, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; PSK; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL®, Bristol Meyers Squibb Oncology, Princeton, N.J.) and docetaxel (TAXOTERE®, Rhone-Poulenc Rorer, Antony, France); chlorambucil; gemcitabine (Gemzar®); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine®); novantrone; teniposide; edatrexate; daunomycin; aminopterin; xeoloda; ibandronate; CPT-11; topoisomerase inhibitor RFS 2000; difluoromethylomithine (DMFO); retinoids such as retinoic acid; capecitabine; FOLFIRI (fluorouracil, leucovorin, and irinotecan) and pharmaceutically acceptable salts, acids or derivatives of any of the above.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston®); inhibitors of the enzyme aromatase, which regulates estrogen production in the adrenal glands, such as, for example, 4(5)-imidazoles, aminoglutethimide, megestrol acetate (Megace®), exemestane, formestane, fadrozole, vorozole (Rivisor®), letrozole (Femara®), and anastrozole (Arimidex®); and anti-androgens such as flutamide, nilutamide, bicalutamide, leuprohde, and goserelin; and pharmaceutically acceptable salts, acids or derivatives of any of the above.

The anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloprotemase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,I-3,4-dehydroproline, thiaproline, .alpha.-dipyridyl, beta-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpba-2-antiplasmin, bisantrene, lobenzarit disodium, n-2-carboxyphenyl-4-chloroanthronilic acid disodium or “CCA”, thalidomide; angiostatic steroid, cargboxynaminolmidazole; metalloproteinase inhibitors such as BB94. Other anti-angiogenesis agents include antibodies, preferably monoclonal antibodies against these angiogenic growth factors: beta-FGF, alpha-FGF, FGF-5, VEGF isoforms, VEGF-C, HGF/SF and Ang-1/Ang-2. See Ferrara N. and Alitalo, K. “Clinical application of angiogenic growth factors and their inhibitors” (1999) Nature Medicine 5:1359-1364.

The anti-fibrotic agents include, but are not limited to, the compounds such as beta-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. No. 5,021,456; U.S. Pat. No. 5,5059,714; U.S. Pat. No. 5,120,764; U.S. Pat. No. 5,182,297; U.S. Pat. No. 5,252,608 (relating to 2-(1-naphthyloxymemyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. Also, the anti-fibrotic agents are copper chelating agents, penetrating or not penetrating the cells. Exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, sodium-4-mercaptobutanesulphinate trihydrate.

The immunotherapeutic agents include and are not limited to therapeutic antibodies suitable for treating patients; such as abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8. The exemplified therapeutic antibodies may be further labeled or combined with a radioisotope particle, such as indium In 111, yttrium Y 90, iodine I-131.

In a certain embodiments, the additional therapeutic agent (e.g., administered in further combination with the PI3K inhibitor and the BTK inhibitor as described herein) is a nitrogen mustard alkylating agent. Nonlimiting examples of nitrogen mustard alkylating agents include chlorambucil.

Some chemotherapy agents suitable for treating lymphoma or leukemia include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, ABT-199, ABT-737, BMS-345541, bortezomib (Velcade®), bryostatin 1, busulfan, carboplatin, campath-1H, CC-5103, carmustine, caspofungin acetate, clofarabine, cisplatin, Cladribine (Leustarin), Chlorambucil (Leukeran), Curcumin, cyclosporine, Cyclophosphamide (Cyloxan, Endoxan, Endoxana, Cyclostin), cytarabine, denileukin diftitox, dexamethasone, DT PACE, docetaxel, dolastatin 10, Doxorubicin (Adriamycin®, Adriblastine), doxorubicin hydrochloride, enzastaurin, epoetin alfa, etoposide, Everolimus (RAD001), fenretinide, filgrastim, melphalan, mesna, Flavopiridol, Fludarabine (Fludara), Geldanamycin (17-AAG), ifosfamide, irinotecan hydrochloride, ixabepilone, Lenalidomide (Revlimid®, CC-5013), lymphokine-activated killer cells, melphalan, methotrexate, mitoxantrone hydrochloride, motexafin gadolinium, mycophenolate mofetil, nelarabine, oblimersen (Genasense) Obatoclax (GX15-070), oblimersen, octreotide acetate, omega-3 fatty acids, oxaliplatin, paclitaxel, PD0332991, PEGylated liposomal doxorubicin hydrochloride, pegfilgrastim, Pentstatin (Nipent), perifosine, Prednisolone, Prednisone, R-roscovitine (Selicilib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, Styryl sulphones, tacrolimus, tanespimycin, Temsirolimus (CCl-779), Thalidomide, therapeutic allogeneic lymphocytes, thiotepa, tipifamib, Velcade® (bortezomib or PS-341), Vincristine (Oncovin), vincristine sulfate, vinorelbine ditartrate, Vorinostat (SAHA), vorinostat, and FR (fludarabine, rituximab), CHOP (cyclophosphamide, doxorubicin, vincristine, prednisone), CVP (cyclophosphamide, vincristine and prednisone), FCM (fludarabine, cyclophosphamide, mitoxantrone), FCR (fludarabine, cyclophosphamide, rituximab), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), R-CHOP (rituximab plus CHOP), R-CVP (rituximab plus CVP), R-FCM (rituximab plus FCM), R-ICE (rituximab-ICE), and R-MCP (R-MCP).

### Methods of Treating Subjects Resistant to Idelalisib

In certain aspects, provided herein is a method for treating B-cell malignancy in a human in need thereof who is resistant, or is developing resistance, to idelalisib, comprising administering to the human a therapeutically effective amount of idelalisib and a therapeutically effective amount of an additional agent. In other aspects, provided herein is a method for treating B-cell malignancy in a human in need thereof to delay or prolong resistance to idelalisib, comprising administering to the human a therapeutically effective amount of idelalisib and a therapeutically effective amount of an additional agent.

In some embodiments of the foregoing aspects, the B-cell malignancy is diffuse large B-cell lymphoma (DLBCL). In one embodiment, the B-cell malignancy is activated B-cell like diffuse large B-cell lymphoma (ABC-DLBCL). In some variations of the foregoing aspects and embodiments, the additional agent is MK-2206 or GSK-2334470. A skilled artisan would recognize that MK-2206 is a Akt inhibitor and GSK-2334470 is a PDK1 inhibitor, with structures known in the art.

In other embodiments of the foregoing aspects, the B-cell malignancy is follicular lymphoma (FL). In some variations of the foregoing embodiment, the additional agent is BYL-719, Dasatinib, or Entospletinib. A skilled artisan would recognize that BYL-719 is a PI3Kα inhibitor; Dasatinib is a Bcr-Abl tyrosine kinase inhibitor and Src family tyrosine kinase inhibitor; and Entospletinib is a Syk inhibitor, with structures known in the art.

### Articles of Manufacture and Kits

Compositions (including, for example, formulations and unit dosages) comprising a PI3K inhibitor, as described herein, and compositions comprising a BTK inhibitor, as described herein, can be prepared and placed in an appropriate container, and labeled for treatment of an indicated condition. Accordingly, provided is also an article of manufacture, such as a container comprising a unit dosage form of a PI3K inhibitor and a unit dosage form of a BTK inhibitor, as described herein, and a label containing instructions for use of the compounds. In some embodiments, the article of manufacture is a container comprising (i) a unit dosage form of a PI3K inhibitor, as described herein, and one or more pharmaceutically acceptable carriers, adjuvants or excipients; and (ii) a unit dosage form of a BTK inhibitor, as described herein, and one or more pharmaceutically acceptable carriers, adjuvants or excipients. In one embodiment, the unit dosage form for both the PI3K inhibitor and the BTK inhibitor is a tablet.

In other aspects, provided is also an article of manufacture, such as a container comprising a unit dosage form of idelalisib and a unit dosage form of MK-2206, GSK-2334470, BYL-719, Dasatinib, or Entospletinib, and a label containing instructions for use of the compounds. In some embodiments, the article of manufacture is a container comprising (i) a unit dosage form of idelalisib, and one or more pharmaceutically acceptable carriers, adjuvants or excipients; and (ii) a unit dosage form of MK-2206, GSK-2334470, BYL-719, Dasatinib, or Entospletinib, and one or more pharmaceutically acceptable carriers, adjuvants or excipients.

Kits also are contemplated. For example, a kit can comprise unit dosage forms of (i) a PI3K inhibitor, as described herein, and (ii) a BTK inhibitor, as described herein, and a package insert containing instructions for use of the composition in treatment of a medical condition. In some embodiments, the kits comprises (i) a unit dosage form of the PI3K inhibitor, as described herein, and one or more pharmaceutically acceptable carriers, adjuvants or excipients; and (ii) a unit dosage form of a BTK inhibitor, as described herein, and one or more pharmaceutically acceptable carriers, adjuvants or excipients. In one embodiment, the unit dosage form for both the PI3K inhibitor and the BTK inhibitor is a tablet.

In other aspects, provided is a kit that comprises unit dosage forms of (i) idelalisib, and (ii) MK-2206, GSK-2334470, BYL-719, Dasatinib, or Entospletinib, and a package insert containing instructions for use of the composition in treatment of a medical condition. In some embodiments, the kits comprises (i) a unit dosage form of idelalisib, and one or more pharmaceutically acceptable carriers, adjuvants or excipients; and (ii) a unit dosage form of MK-2206, GSK-2334470, BYL-719, Dasatinib, or Entospletinib, and one or more pharmaceutically acceptable carriers, adjuvants or excipients.

The instructions for use in the kit may be for treating a B-cell malignancy as further described herein.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example 1A: Growth Inhibition Assay in DLBCL Cell Lines

This example evaluates the anti-proliferative activity of Idelalisib in combination with Compound B in three DLBCL cell lines.

### Materials and Methods

Cell Lines and Culture Conditions:

The combination of Idelalisib (referred to as Compound A) and a monohydrochloride salt of 6-amino-9-[(3R)-1-(2-butynoyl)-3-pyrrolidinyl]-7-(4-phenoxyphenyl)-7,9-dihydro-8H-purin-8-one (referred to in the Examples as Compound B) was evaluated in an in vitro growth inhibition assay in three ABC-DLBCL cell lines (OCI-LY10, Ri-1, and TMD-8) and 1 GCB-DLBCL cell line (Pfeiffer). Other DLBCL cell lines, including NU-DUL-1, SU-DUL-8, SU-DHL-2, OCI-Ly3 and U-2932 were also tested for growth inhibition assays with treatment of Idelalisib, Compound B and ibrutinib.

Cell lines were obtained from American Type Culture Collection (ATCC), Leibniz-Institut DSMZ-Deutsche Sammlung von Mikrooorgansimen und Zellkulturen GmbH (DSMZ), University Health Network (Toronto, CA) or the Tokyo Medical and Dental Institute. Cells were cultured according to instructions provided. The complete culture medium was prepared using RPMI base medium (Gibco cat. no. 22400-089) supplemented with 20% heat-inactivated fetal bovine serum (Gibco cat no. 16140-063) and 100 U/L penicillin-streptomycin (Gibco cat no. 15140-148). Cells were incubated at 37° C./5% CO₂. See Table A below.

Genetic Mutation Profiling: Mutations in the components in common signaling pathways in the cell lines used were determined using the FoundationOne® Heme assay (Foundation Medicine).

TABLE A
Summary of DLBCL cell lines used

| Cell Line | Lymphoma | | |
|---|---|---|---|
| Name | Subtype | Source | Growth Medium |
| TMD-8 | ABC-DLBCL | Tokyo Medical and | RPMI + 20% FBS |
| | | Dental | |
| OCI-LY10 | ABC-DLBCL | University Health | RPMI + 20% FBS |
| | | Network | |
| | | Toronto, Canada | |
| Ri-l | ABC-DLBCL | DSMZ | RPMI + 20% FBS |
| Pfeiffer | GCB-DLBCL | ATCC | RPMI + 20% FBS |

Cell Viability Assay:

In vitro anti-proliferative activity of agents was assessed using CellTiter-Glo™ Assay (Promega), which quantifies cellular ATP level. Test compounds were dissolved in DMSO to prepare 10 mM stock solutions. For single agent EC₅₀ determinations, all test compounds were serially diluted three fold with DMSO in a 96 well plate to achieve a final dose range of 10 μM-0.51 nM in a solution of 0.1% DMSO in the test medium. For drug combination studies, a two or three fold horizontal serial dilution pattern was used for Compound B and combined with Idelalisib using a two, three, or four fold vertical serial dilution pattern. The highest concentration tested varied based on the EC₅₀ of the cell line with the maximal concentration of 10 μM. The final DMSO concentration in the test media was 0.2%. Each combination used replicates of four plates to generate sufficient data for evaluation of synergy scores. All test plates contained one column each of control wells representing 0% inhibition (DMSO) and 100% inhibition (2 μM staurosporine). The assay growth medium for all lines was RPMI supplemented with 20% FBS and 100 U/L penicillin-streptomycin. Seeding density was optimized for growth rate over 96 hours for each cell line and was between 10,000-30,000 cells per well of 96 well plates. After four days incubation with agents at 37° C./5% CO₂, the CellTiter-Glo™ assay was performed following the manufacturer's protocol. Relative luminescence units were quantified using a Biotek Synergy luminometer.

Data Analysis:

Data from a plate was excluded if either Z′<0.5 or the growth of the cell lines was less than one doubling in the four day assay period. Z′ was calculated using the formula: Z′=[1−((3(σstau+σ_{DMSO}))/(|μ_{DMSO}−μₛₜₐᵤ|)] where σₛₜₐᵤ and σ_{DMSO} are the standard deviation of the 100% inhibited staurosporine and 0% inhibited DMSO control wells, respectively. μ_{DMSO} and μₛₜₐᵤ are the mean of the 100% inhibited staurosporine and 0% inhibited DMSO control wells, respectively. The raw Cell Titer Glo signal was normalized according to the following formula: [(raw value)−(100% inhibited staurosporine control)]/[(DMSO control value)−(100% inhibited staurosporine control)].

EC₅₀ was determined using GraphPad Prism or Dose Response software by fitting the data to a four parameters variable slope model. EC₉₀ was calculated by fitting the data using the “Find ECanything” variable slope model and setting F to 10. Eₘₐₓ at 10 μM was determined by taking the ratio of the signal at 10 μM inhibitor to the signal from the no inhibitor control. For combination studies, the EC₅₀ at each test article concentration was determined from graphs of the EC₅₀ of one compound at a fixed dose of second compound. The EC₅₀ shift was calculated by taking the ratio of the EC₅₀ of the single agent by the EC₅₀ at the maximum dose of the second agent.

Synergy was analyzed using the MacSynergy II program, which calculates a theoretical additive value for the drug combination that is based on the values generated by each drug alone using the Bliss Independence mathematical model. The Bliss independence model assumes that each drug acts independently. The theoretical additive effect for each compound is calculated and then subtracted from the actual effect. Synergy is defined by greater than expected effects while antagonism is defined by less than expected effects. In this example, a synergy volume greater than 50 was considered significant. The EC₅₀ values determined in drug combination studies represent a single experiment run in quadruplicate and therefore may differ slightly from the single agent EC₅₀ values.

Apoptosis Assay:

Apoptosis of Idelalisib in combination with Compound B in two DLBCL cell lines, OCI-Ly10 and TMD-8, was also measured. Cells plated at 0.2×10⁶ cells/mL in RPMI 1640 supplemented with 20% RPMI and 1% penicillin and streptomycin. Cells were treated with compound, 156 nM Idelalisib, Compound B and the combination thereof. Control received DMSO at 0.2%. Cells were then incubated at 37 C for 48 hours. Apoptosis was measured using Annexin V/FITC kit, and analyzed by flow cytometry. Apoptosis was also measured using or Annexin V/7ADD kit (Beckman Coulter). Fluorescence was measured by flow cytometry using BD LSRII and the results were analyzed using FACSDiva.

### Results

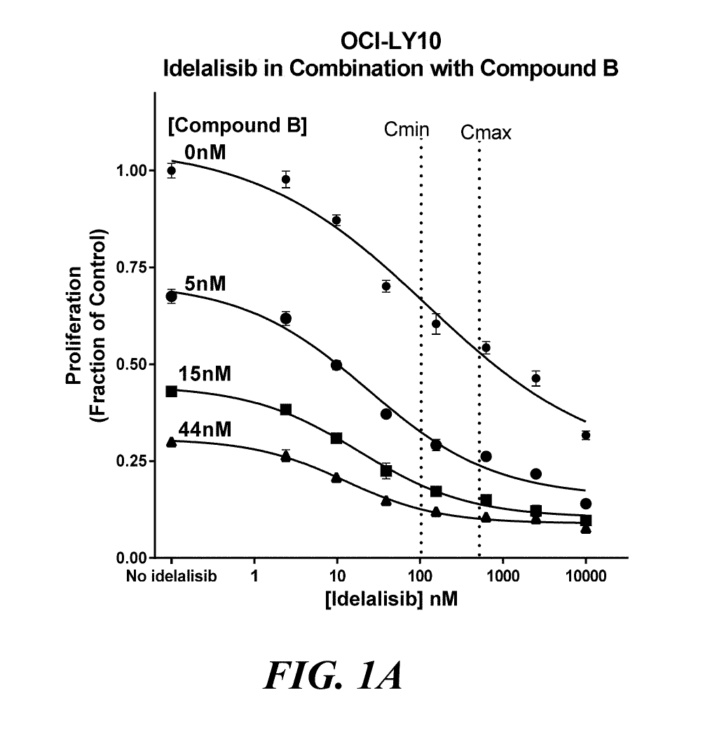

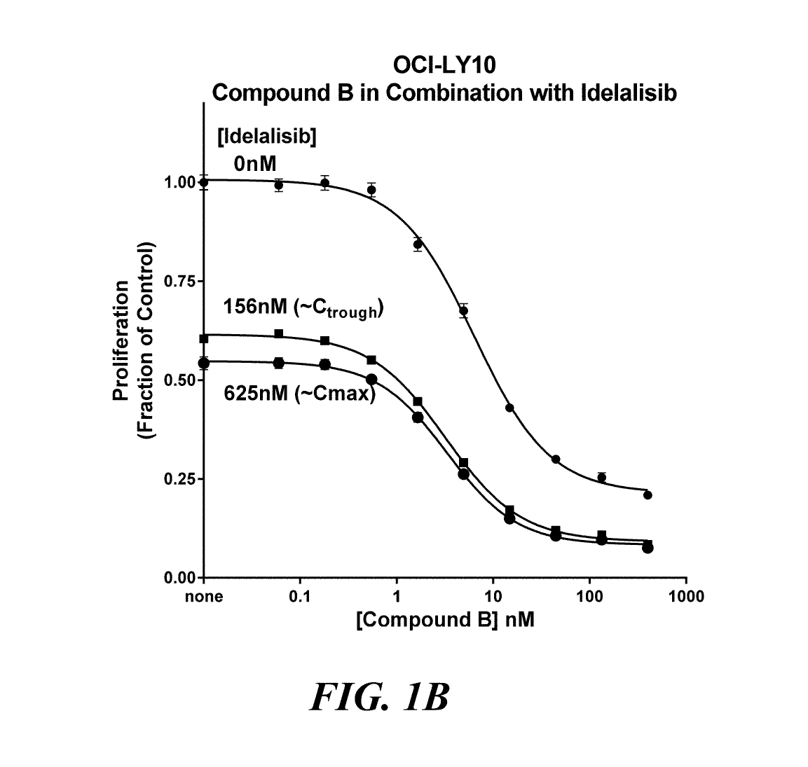

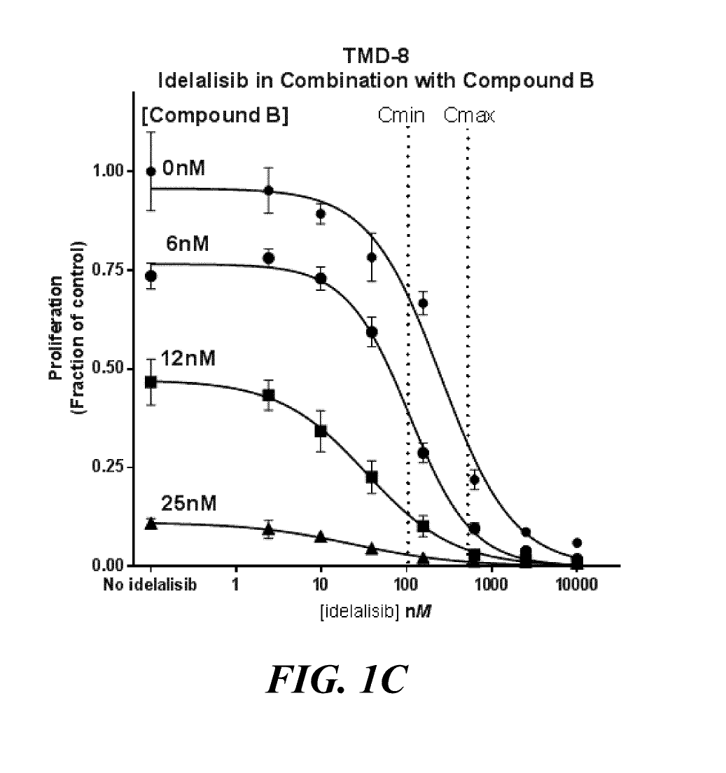

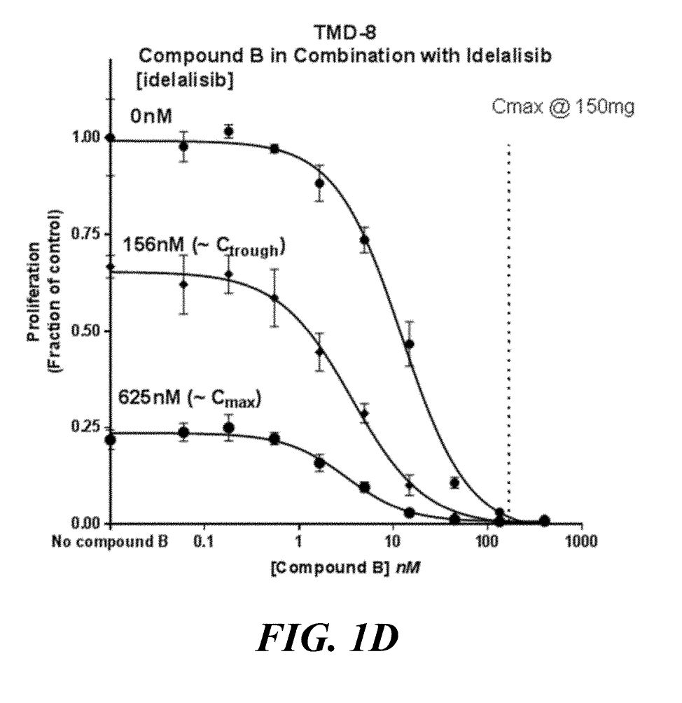

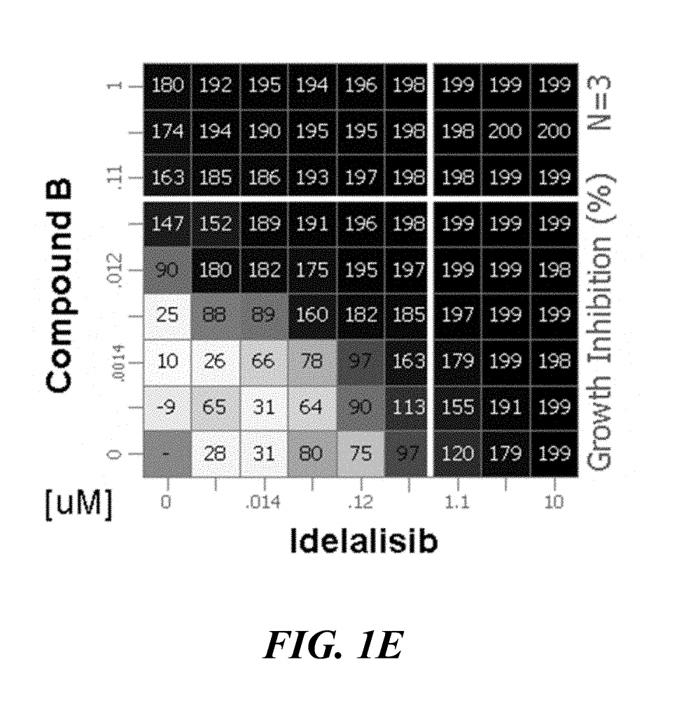

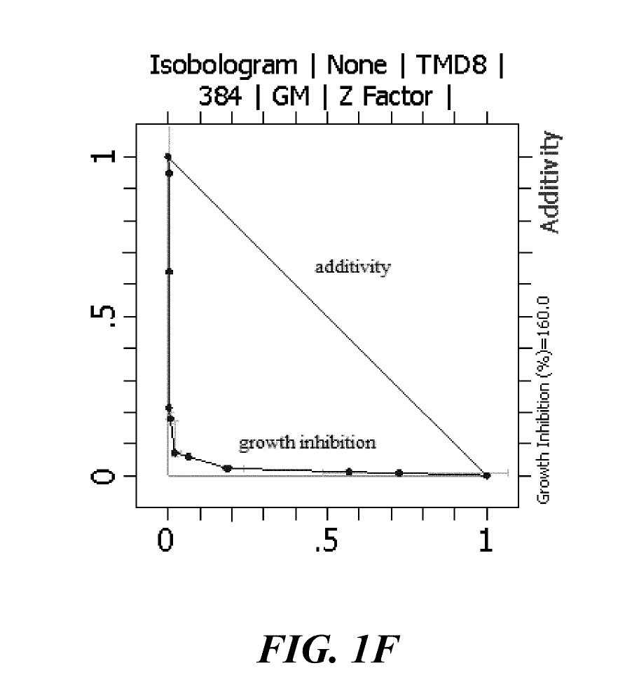

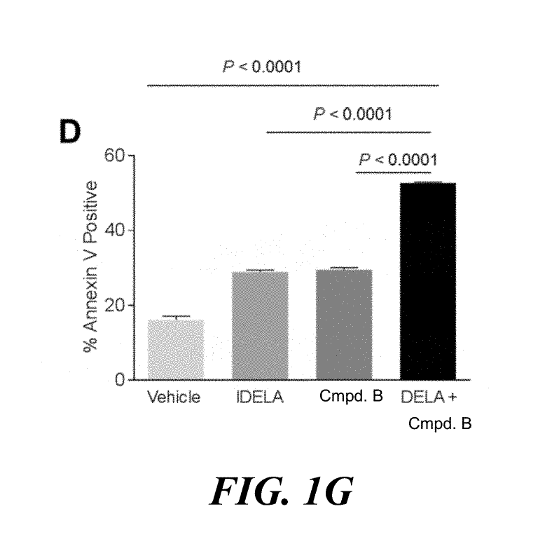

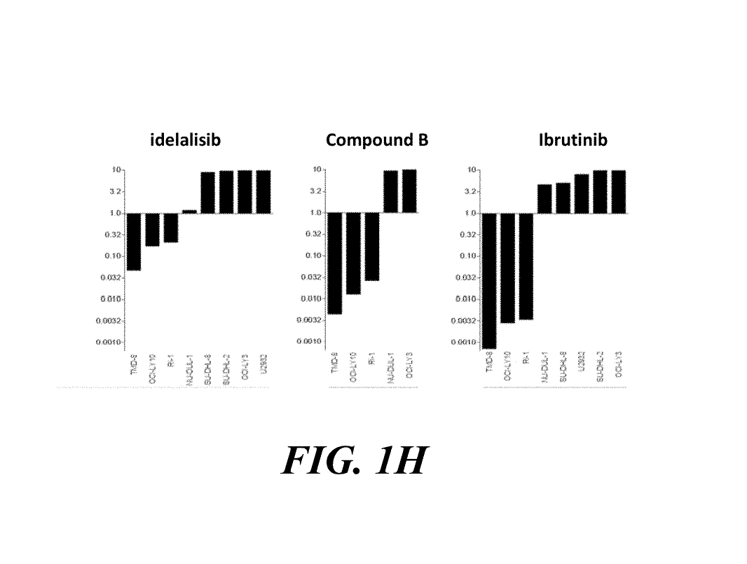

Compound B was observed to potently inhibit growth (EC50<26 nM) of three ABC-DLBCL cell lines (OCI-LY10, Ri-1, and TMD-8) that were also sensitive to Idelalisib (EC50<210 nM). The combination of Idelalisib and Compound B showed synergistic growth inhibition in ABC-DLBCL cell lines OCI-LY10 and TMD-8 and increased apoptosis above the level observed with single agents as shown in FIGS. 1A-1D and Tables 1-3 below. Additional results are shown in FIGS. 1G.

Idelalisib, Compound B and Ibrutinib inhibited the growth of OCI-LY10, Ri-1, and TMD-8 cell lines. The idelalisib concentrations used in the experiments represented clinically relevant ranges: 103 and 591 nM corresponded to clinical Cmin and Cmax, respectively. A synergistic effect in combination with Compound B on cell viability in TMD8 and OCI-LY10 was observed. The addition of Compound B at 6, 12, and 25 nM to idelalisib in the TMD8 cell line shifted the EC50 values from 254 nM to 108, 34 and 24 nM, respectively and in OCI-LY10 cell line shifted the EC50 values from 122 nM to 24, 19, and 13 nM, respectively.

Analysis of mutations in common signaling pathway components showed that the OCI-LY10, Ri-1, and TMD-8 cell lines did not have a mutation in PI3KCA, AKT1/AKT2, TP53 or PTEN genes. Additionally, the results shown that TMD-8 and OCI-LY10 contained mutations in CD79A/CD79B and MYD88; that Ri-1 contained mutation in TP53 and amplifications in AKT1/AKT2 and MALT1; that NU-DUL-1 and SU-DUL-8 contained mutation in TP53; that OCI-LY3 contained mutations in CD79A/CD79B, CARD11, and MYD88, deletion in TP53, and amplification in RB1, and that U-2932 contained mutation in TP53, amplification in MALT1, and deletion in RB1.

TABLE 1
Shift in Idelalisib EC₅₀ in Combination with Compound B

| | EC₅₀^{a} of Idelalisib (nM) when Combined with | | | |
|---|---|---|---|---|
| Compound B | Compound B | | | |
| (nM) | TMD-8 | OCI-LY-10 | Ri-l | Pfeiffer |
| 0 | 254 | 440 | 442 | 174 |
| 5 | 130 | 38 | 372 | NT^{c} |
| 15 | 32 | 22 | 372 | NT |
| 45 | 24 | 5 | 372 | 174 |
| EC₅₀ shift (fold) | 10.6 | 88 | 1.2 | 1 |
| Synergy Score^{b} | 65 | 65 | 0 | 0 |

^{a}Concentration of inhibitor eliciting a 50% effect on cellular viability
^{b}Bliss synergy score: >50 is considered synergistic
^{c}Not tested

TABLE 2
Shift in Compound B EC₅₀ in Combination with Idelalisib

| | EC₅₀^{a} of Compound B in Combination with | | | |
|---|---|---|---|---|
| Idelalisib | Idelalisib (nM) | | | |
| (nM) | TMD-8 | OCI-LY-10 | Ri-l | Pfeiffer |
| 0 | 11 | 6 | 5 | >10 |
| 156 | 5 | 3 | 2 | >10 |
| 625 | 4 | 3 | 2 | >10 |
| EC₅₀ shift (fold) | 2.75 | 2 | 2.5 | None^{c} |

^{a}Concentration of inhibitor eliciting a 50% effect on cellular viability
^{c}No EC₅₀ shift observed at up to 2.5 μM

TABLE 3
Annexin V positivity calculated by flow cytometry at 48 h,
Idelalisib = 156 nM, Compound B = 8 nM

| | | | | Idela + |
|---|---|---|---|---|
| % Annexin V+ | DMSO | Idelalisib | Compound B | Compound B |
| TMD-8 | 21 | 45 | 44 | 64 |
| OCI-Ly10 | 29 | 25 | 26 | 54 |

### Example 1B: Cell Viability Assay in TMD-8

The effects of administering Idelalisib in combination with Compound B in TMD-8 cell line described above were further explored in this example.

Anti-Proliferation Assay:

The endpoint readout of the anti-proliferation assay was based upon quantitation of ATP as an indicator of viable cells. Cells were thawed from a liquid nitrogen preserved state. Once cells had been expanded and divided at their expected doubling times, screening began. Cells were seeded in growth media in black 384-well tissue culture treated plates at 500 cells per well (except where noted in Analyzer). Cells were equilibrated in assay plates via centrifugation and placed in incubators attached to the Dosing Modules at 37° C. for twenty-four hours before treatment. At the time of treatment, a set of assay plates (which did not receive treatment) were collected and ATP levels were measured by adding ATPLite (Perkin Elmer). These Tzero (T₀) plates were read using ultra-sensitive luminescence on Envision Plate Readers. Treated assay plates were incubated with the compound for one hundred twenty hours. After one hundred twenty hours, plates were developed for endpoint analysis using ATPLite. All data points were collected via automated processes; quality controlled; and analyzed using Horizon CombinatoRx proprietary software. Assay plates were accepted if they pass the following quality control standards: relative luciferase values were consistent throughout the entire experiment, Z-factor scores were greater than 0.6, untreated/vehicle controls behaved consistently on the plate.

Horizon Discovery utilized Growth Inhibition (GI) as a measure of cell viability. The cell viability of vehicle was measured at the time of dosing (T₀) and after one hundred twenty hours (T₁₂₀). A GI reading of 0% represented no growth inhibition—cells treated with compound and T₁₂₀ vehicle signals were matched. A GI 100% represents complete growth inhibition—cells treated by compound and T₀ vehicle signals were matched. Cell numbers had not increased during the treatment period in wells with GI 100% and may suggest a cytostatic effect for compounds reaching a plateau at this effect level. A GI 200% represents complete death of all cells in the culture well. Compounds reaching an activity plateau of GI 200% were considered cytotoxic. Horizon CombinatoRx calculates GI by applying the following test and equation:

$$If T < {V}_{0} \text{:} 100 * ( 1 - \frac{T - {V}_{0}}{{V}_{0}} )$$

$$If T \geq {V}_{0} \text{:} 100 * ( 1 - \frac{T - {V}_{0}}{V - {V}_{0}} )$$

where T is the signal measure for a test article, V is the vehicle-treated control measure, and Vₒ is the vehicle control measure at time zero. This formula was derived from the Growth Inhibition calculation used in the National Cancer Institute's NCI-60 high throughput screen.

Synergy Score Analysis:

To measure combination effects in excess of Loewe additivity, Horizon Discovery has devised a scalar measure to characterize the strength of synergistic interaction termed the Synergy Score. The Synergy Score was calculated as:

Synergy Score=log fx log fyΣmax(0,I_{data})(I_{data}−I_{Loewe})

The fractional inhibition for each component agent and combination point in the matrix was calculated relative to the median of all vehicle-treated control wells. The Synergy Score equation integrated the experimentally-observed activity volume at each point in the matrix in excess of a model surface numerically derived from the activity of the component agents using the Loewe model for additivity. Additional terms in the Synergy Score equation (above) were used to normalize for various dilution factors used for individual agents and to allow for comparison of synergy scores across an entire experiment. The inclusion of positive inhibition gating or an I_{data} multiplier removed noise near the zero effect level, and biased results for synergistic interactions at that occur at high activity levels.

Potency shifting was evaluated using an isobologram, which demonstrates how much less drug is required in combination to achieve a desired effect level, when compared to the single agent doses needed to reach that effect. The isobologram was drawn by identifying the locus of concentrations that correspond to crossing the indicated inhibition level. This was done by finding the crossing point for each single agent concentration in a dose matrix across the concentrations of the other single agent. Practically, each vertical concentration C_{Y} was held fixed while a bisection algorithm was used to identify the horizontal concentration C_{X} in combination with that vertical dose that gives the chosen effect level in the response surface Z(C_{X},C_{Y}). These concentrations were then connected by linear interpolation to generate the isobologram display.

For synergistic interactions, the isobologram contour falls below the additivity threshold and approaches the origin, and an antagonistic interaction would lie above the additivity threshold. The error bars represent the uncertainty arising from the individual data points used to generate the isobologram. The uncertainty for each crossing point was estimated from the response errors using bisection to find the concentrations where Z-σσ_{Z}(C_{X},C_{Y}) and Z+σ_{Z}(C_{X},C_{Y}) cross I_{cut}, where σ_{Z} is the standard deviation of the residual error on the effect scale.

### Results

FIG. 1E visually depicts the cell death effects of administering the combination of Idelalisib and Compound B, and FIG. 1F is the isobologram generated from the data in this example. The synergy score for the assay performed in this example was observed to be 44. The assay performed in this example had a range of 0.2-44. Thus, the observed score of 44 demonstrated synergy for the combination of Idelalisib and Compound B.

### Example 2: Dose Escalation Study

This example evaluates the safety, tolerability, PK, pharmacodynamics, and preliminary efficacy of Compound B in combination with Idelalisib in subjects with B-cell lymphoproliferative malignancies. Subjects with B-cell malignancies who have refractory or relapsed disease are sequentially enrolled at progressively higher dose levels to receive oral Compound B combined with Idelalisib.

The starting dose of Compound B is 20 mg once daily and of Idelalisib is 50 mg twice daily. If a dose-limiting toxicity (DLT) occurs within 28 days from Cycle 1, Day 1 in Cohort 1A, this cohort will be expanded to enroll 3 additional subjects. If ≥2 DLTs occur in Cohort 1A, development of the combination of Compound B and Idelalisib will discontinue. If no DLT occurs in 3 subjects or <2 DLTs occur in up to 6 subjects in Cohort 1A, then Cohort 2A will open. Cohort 2A will enroll 3 subjects with Compound B dosed at 40 mg once daily and Idelalisib 50 mg twice daily. Once enrollment is complete in Cohort 2A, Cohort 2B will enroll 3 subjects with Compound B dosed at 20 mg twice daily and Idelalisib 50 mg twice daily. Cohorts 2A and 2B will dose escalate independently and in parallel; if no DLT occurs in 3 subjects or <2 DLTs occur in up to 6 subjects in Cohort 2A and Cohort 2B has completed enrollment, then the next 3 subjects will be enrolled in Cohort 3A with Compound B dosed at 80 mg once daily and Idelalisib 50 mg twice daily. Similarly, if no DLT occurs in 3 subjects or <2 DLTs occur in up to 6 subjects at Cohort 2B, Cohort 3B will enroll 3 subjects with Compound B dosed 40 mg twice daily and Idelalisib 50 mg twice daily. Subsequent cohorts will enroll if no DLTs in 3 subjects or <2 DLTs occur in up to 6 subjects are observed. If a second DLT is observed in any cohort, maximum tolerated dose (MTD) of Compound B combined with Idelalisib will have been exceeded and the prior cohort will be the MTD. The MTD for Compound B once-daily will be determined separately from the MTD for Compound B twice-daily.

All available safety, tolerability, and PK data will be reviewed upon completion of the above described protocol.

Once the MTD of the combination of Compound B with 50 mg of Idelalisib twice daily has been determined, based on safety and efficacy, 1 additional cohort may be enrolled at up to 50% of the MTD of Compound B combined with 100 mg of Idelalisib twice daily. The doses for each cohort are shown in Table 4.

TABLE 4
Cohort doses

| | |
|---|---|
| | Compound B |
| | (mg) |
| Dose | (Combined with Compound A 50 mg BID) |

| Level | Cohort A | Cohort B |
|---|---|---|
| 1 | 20 QD | — |
| 2 | 40 QD | 20 BID |
| 3 | 80 QD | 40 BID |
| 4 | 150 QD | 75 BID |

QD = Once daily dosing,
BID = Twice daily dosing

DLT:

A DLT is a toxicity defined below considered possibly related to Idelalisib and/or Compound B, and occurs during the DLT assessment window (Day 1 through Day 29) in each cohort:

1) All Grade ≥4 hematological toxicities persisting for >7 days

2) All Grade ≥3 non-hematological toxicities (except for alopecia, or nausea, vomiting, diarrhea, or constipation that resolves within 72 hours with medical intervention)

3) All Grade ≥4 laboratory abnormalities

4) Febrile Neutropenia (defined as ANC <1.0×10₉/L with a single temperature >38.3° C. [101 F] or sustained temperature ≥38 C [100.4 F] for more than 1 hour)

5) Grade ≥2 non-hematologic treatment-emergent adverse event (TEAE) that in the opinion of the investigator is of potential clinical significance such that further dose escalation would expose subjects to unacceptable risk.

Treatment:

Subjects who meet eligibility criteria will receive a single dose of Compound B on Cycle 1, Day 1 and then initiate Idelalisib in combination with Compound B on Cycle 1, Day 2. The first cycle will consist of 28 days (1 day of single agent Compound B and 27 days of combination treatment), and each subsequent cycle will be 28 days of combination treatment. Safety and efficacy assessments will occur on an outpatient basis including assessment of tumor response, physical exam, vitals, ECG, collection of blood samples (for routine safety labs, Compound B and Idelalisib PK, pharmacodynamics, and biomarkers at applicable visits), and assessment of adverse events (AEs) (e.g., diarrhea/colitis, transaminase elevation, rash, and pneumonitis). In addition, subjects will undergo a CT (or MRI) scan every 12 weeks (6 weeks for DLBCL for the first 12 weeks). A subject who does not show evidence of disease progression by clinical assessment or by CT (or MRI) may continue receiving Compound B in combination with Idelalisib daily until disease progression (clinical or radiographic), unacceptable toxicity, withdrawal of consent, or other reasons. After discontinuation of treatment, subjects will be followed for safety for 30 days.

PK and Pharmacodynamics Sampling:

PK samples will be collected on Cycle 1, Day 1 at pre-dose and 0.5, 1, 2, 3, 4, 6, 8, and 12 hours (optional) post-dose of Compound B and Cycle 1, Days 2 and 8 at pre-dose and 0.5, 1, 2, 3, 4, 6, 8, 12 (optional), and 24 hours post-dose of Compound B and Idelalisib. The 12 hour post-dose PK samples are optional. The 12-hour post-dose PK sample should be collected prior to evening dose when study drug is administered BID and 24 hour sample will be collected 24 hours post-dose relative to morning dose. PK samples will be collected in all cohorts at pre-dose and 1-6 hours post-dose on Cycle 1 Day 15. A sparse PK sample will also be collected anytime on the first day of Cycles 2 to 6. Blood samples for pharmacodynamics will be collected on Cycle 1, Day 1 at pre-dose, and 1, 2, 4, and 6 hours post-dose and at pre-dose, and 1, 2, 4, 6 and 24 hours post-dose on Cycle 1, Days 2 and 8. The collection of some or all of these samples may not be feasible at the site due to shipment logistics depending on their geographic location. In addition, sampling time points may be eliminated or modified based upon emerging data.

Dose and Mode of Administration:

Compound B will be self-administered orally once or twice daily depending on cohort, beginning on Cycle 1, Day 1 of the study and thereafter at approximately the same time each day until end of treatment. Idelalisib will be self-administered orally twice daily, beginning on Cycle 1, Day 2 and at the same time as (within 10 minutes of) Compound B. Compound B is supplied as 10 and 25 mg capsules. Idelalisib is supplied as 50 mg and 100 mg tablets.

The dosing regimen of the combination of Compound B and Idelalisib for use in future clinical trials in subjects with FL, MZL, CLL, SLL, MCL, WM, and non-GCB-DLBCL will be chosen based on safety and efficacy data supported by PK and pharmacodynamics data.

### Example 3A: Growth Inhibition Assay in MCL Cell Lines

This example evaluates the anti-proliferative activity of Idelalisib in combination with Compound B in various MCL cell lines.

### Materials and Methods

Cell Lines and Culture Conditions:

The combination of Idelalisib and a monohydrochloride salt of 6-amino-9-[(3R)-1-(2-butynoyl)-3-pyrrolidinyl]-7-(4-phenoxyphenyl)-7,9-dihydro-8H-purin-8-one (referred to in the Examples as Compound B) was evaluated in an in vitro growth inhibition assay in various MCL cell lines, including Rec-1. JVM-2, Granta-519, Jeko-1, JMP-1, JVM-13, Maver-1, Mino, PF-1, PF-2, and Z-138. These cell lines were cultured in accordance with the procedures set forth in Example 1A.

Anti-Proliferation Assay & Synergy Score Analysis:

The cell viability of vehicle was measured at the time of dosing (T₀) and after 120 h (T₁₂₀). GI reading of 0% represents no growth inhibition, GI 100% represents complete growth inhibition, and GI 200% represents complete death of all cells. To measure combination effects, excess of Loewe additivity was used to characterize the strength of synergistic interaction, termed the synergy score. The assay and synergy score analysis were performed in accordance with the protocol set forth in Example 1B above.

### Results

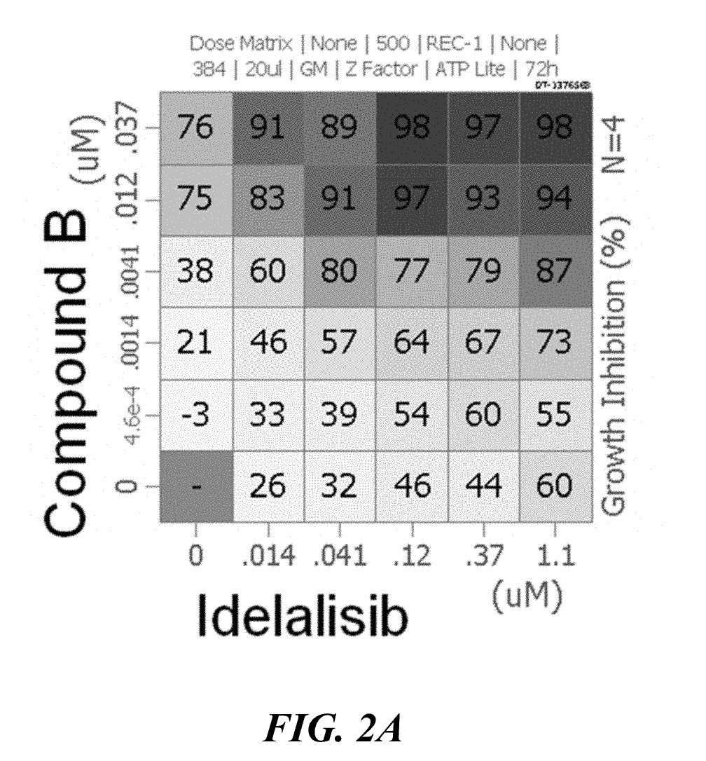

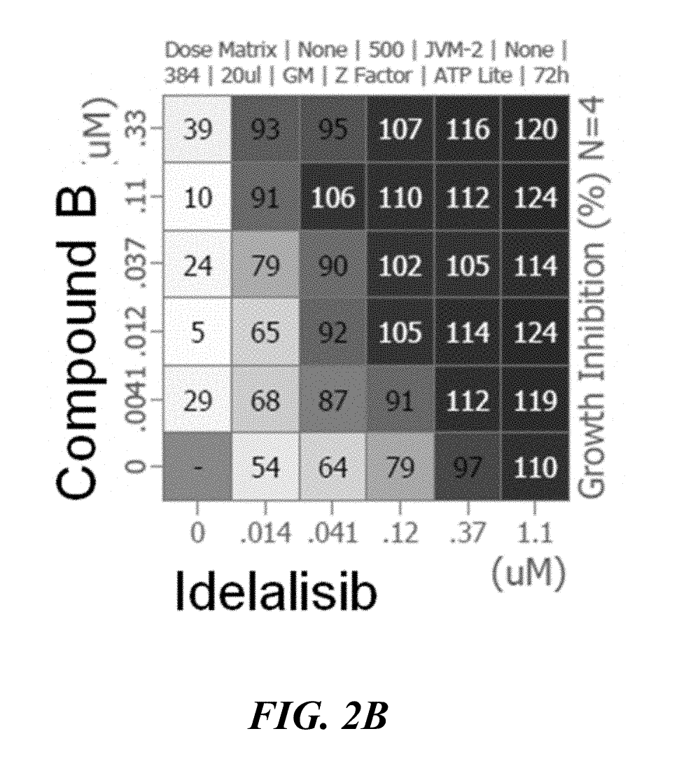

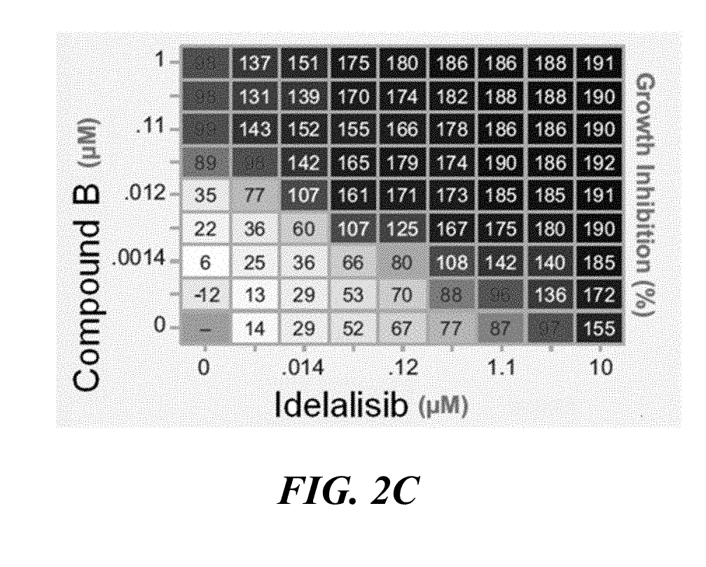

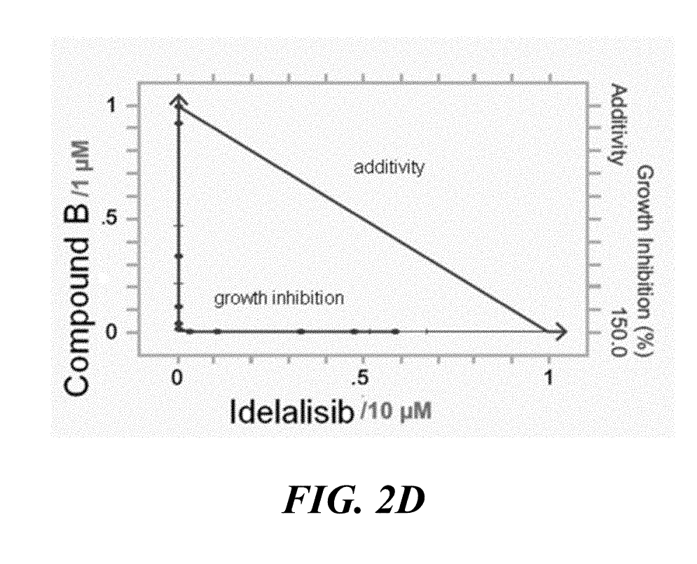

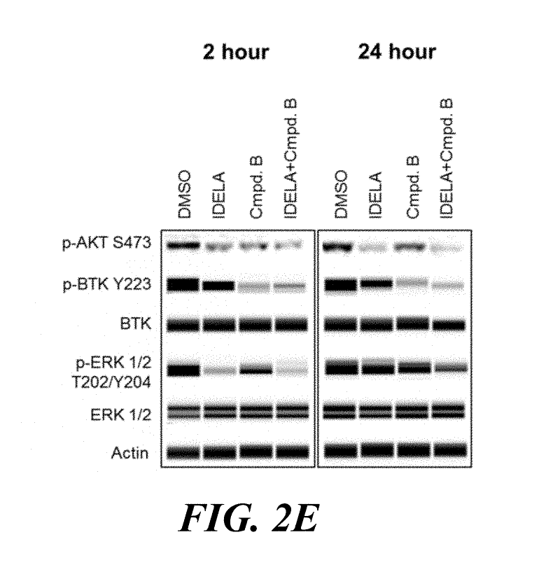

The results of the administration of the combination of Idelalisib and Compound B are summarized in Table 5 below. The administration of the combination of Idelalisib with Compound B was also observed to synergistically inhibit growth in 2 MCL cell lines (Rec-1 and JVM-2). See FIGS. 2A and 2B. For Rec-1, a Synergy Score of 4.1 was observed; and for JVM-2, a Synergy Score of 6.2 was observed. In this example, a synergy score of 4 and above was considered significant, and the synergy range was observed to be 4.0-19.0.

TABLE 5
Summary of Idelalisib and Compound B on MCL Cell Lines

| | Idelalisib | Compound B | Combination |
|---|---|---|---|
| Cell Lines | Max. Effect (% GI) | Max. Effect (% GI) | Synergy Score |
| Granta-519 | 72 | 22 | 0.8 |
| Jeko-1 | 73 | 37 | 1.2 |
| JMP-1 | 69 | 14 | 0.1 |
| JVM-13 | 92 | 15 | 1.3 |
| JVM-2 | 146 | 91 | 6.2 |
| Maver-1 | 48 | 45 | 0.3 |
| Mino | 34 | 35 | 2.5 |
| PF-1 | 29 | 9 | 0 |
| PF-2 | 113 | 25 | 0.5 |
| Rec-1 | 163 | 111 | 4.1 |
| Z-138 | 11 | 11 | 0.1 |

### Example 3B: Growth Inhibition Assay in DLBCL Cell Lines

This example evaluates the anti-proliferative activity of Idelalisib in combination with Compound B in various DLBCL cell lines.

### Materials and Methods

Cell Lines and Culture Conditions:

The combination of Idelalisib and a monohydrochloride salt of 6-amino-9-[(3R)-1-(2-butynoyl)-3-pyrrolidinyl]-7-(4-phenoxyphenyl)-7,9-dihydro-8H-purin-8-one (referred to in the Examples as Compound B) was evaluated in an in vitro growth inhibition assay in various DLBCL cell lines, including HBL-1, OCI-Ly3, Ri-1, SU-DHL-2, TMD-8, U2932, OCI-Ly4, Pfeiffer, SU-DHL-10, SU-DHL-6, SU-DHL-8, Camaval, and U2973. These cell lines were cultured in accordance with the procedures set forth in Example 1A.

Anti-Proliferation Assay & Synergy Score Analysis:

The assay and synergy score analysis were performed in accordance with the protocol set forth in Example 3A above.

Western Blots:

Western Blot samples were prepared by lysing 10⁶ cells for 30 minutes in 150 μL ice-cold lysis buffer. Protease Inhibitor Cocktail (Roche Diagnostics Corp), and phosphatase inhibitor sets 1 and 2 (EMD Millipore) were also added to the lysis buffer (Cell Signaling Technology). Cells were centrifuged at 12.5 g for 10 minutes at 4° C.; supernatant was collected and transferred to new tube. Sample buffer was added to each lysate, and then boiled at 99° C. for 5 minutes. Protein was loaded into an SDS-PAGE gel and run for 2 h at 125V. After electrophoresis, gel was transferred to Immobilon-F membrane using the X Cell Blot. The membrane was then blocked for 1 h at room temperature in blocking buffer and incubated overnight with primary antibodies diluted in a blocking buffer. The antibodies used are indicated in Table 7 below. The following day, the membranes were washed 3× (5 min each) with TBST; secondary antibodies were added and membrane was incubated for 45 minutes at room temperature, followed by 3×TBST (5 min each). Blots were read using Licor Imager. Primary antibodies included p-AKT (S473), p-BTK (Y223), BTK, p-ERK (T202/Y204), ERK, and actin (Cell Signaling Technologies), and secondary antibodies included IRDye-conjugated anti-mouse and anti-rabbit antibodies; LI-COR. Band intensity was measured using LI-COR imager and LI-COR Odyssey software.

Protein Expression Analysis:

Lysates were also analyzed by Simple Western using Peggy Sue (ProteinSimple). A standard curve using recombinant proteins was generated to measure PI3K isoform levels on Peggy Sue; data was processed using Compass software (ProteinSimple).

### Results

The results of the administration of the combination of Idelalisib and Compound B are summarized in Table 6 below. The administration of the combination of Idelalisib with Compound B was also observed to synergistically inhibit growth in several DLBCL cell lines, including TMD-8, U2932 and OCI-Ly4. For TMD-8, a Synergy Score of 65.7 was observed; for U2932, a Synergy Score of 7.9 was observed; and for OCI-Ly4, a Synergy Score of 8.7 was observed. In this example, a synergy score of 6.6 and above was considered significant, and the synergy range was observed to be 6.6-65.7.

TABLE 6
Summary of Idelalisib and Compound B on DLBCL Cell Lines

| | Idelalisib | Compound B | Combination |
|---|---|---|---|
| Cell Lines | Max. Effect (% GI) | Max. Effect (% GI) | Synergy Score |
| HBL-1 | 29 | 65 | 5.8 |
| OCI-Ly3 | 20 | 0 | 0.7 |
| Ri-1 | 78 | 48 | 5.3 |
| SU-DHL-2 | 8 | 18 | 0.3 |
| TMD-8 | 155 | 98 | 65.7 |
| U2932 | 61 | 81 | 7.9 |
| OCI-Ly4 | 194 | 25 | 8.7 |
| Pfeiffer | 33 | 8 | 1.3 |
| SU-DHL-10 | 0 | 0 | 0.08 |
| SU-DHL-6 | 110 | 24 | 2.8 |
| SU-DHL-8 | 43 | 1 | 0.07 |
| Carnaval | 62 | 0 | 0.04 |
| U2973 | 85 | 14 | 1.5 |

FIG. 2C visually depicts the cell death effects of administering the combination of Idelalisib and Compound B, and FIG. 2D is the isobologram generated from the data in this example. The isobologram was generated according to the procedure set forth in Example 1B above.

Table 7 below summarizes the results from TMD-8 Western Blots, taken after 2 and 24 hours. The inhibition of key survival and proliferation pathways was observed in a sustained manner with the combination treatment of Idelalisib and Compound B, as seen below.

TABLE 7
TMD-8 Western Blots

| | | | | | | Idelalisib + |
|---|---|---|---|---|---|---|
| TMD-8 Western Blots | | | DMSO | Idelalisib | Compound B | Compound B |
| pAKT | 2 | hr | 0.45 | 0.19 | 0.24 | 0.16 |
| (Ser 473) | 24 | hr | 0.54 | 0.12 | 0.35 | 0.09 |
| pBTK | 2 | hr | 0.36 | 0.39 | 0.16 | 0.17 |
| (Y223) | 24 | hr | 0.33 | 0.27 | 0.14 | 0.12 |
| pErk1/2 | 2 | hr | 1.09 | 0.40 | 0.47 | 0.32 |
| (T202/Y204) | 24 | hr | 1.49 | 0.55 | 0.93 | 0.32 |

Units are normalized integrated intensity

FIG. 2E depicts results from Western Blots determining the phosphorylation state of signaling pathway components. Idelalisib elicited an increased inhibition to p-AKT (S473) and p-ERK (T202/Y204) (58% and 71%, respectively) than those of Compound B (46% and 48%, respectively). Compound B inhibited BTK activation as measured by p-BTK (Y223) (59%). At 2 hours, the combination of idelalisib and Compound B showed comparable results as those of single agent alone. At 24 hours, the combination of idelalisib and Compound B elicited increased levels of inhibition to p-AKT (S473), p-BTK (Y223) and p-ERK (T202/Y204) (83%, 66% and 36%, respectively) compared to single agent alone.

### Example 4A: BTK Inhibitor Mechanisms of Resistance

### Materials and Methods

Generation of Ibrutinib-Resistant Clones:

To evaluate mechanisms of Ibrutinib resistance in TMD8, several independent clonal isolates of TMD8 were generated through 2 rounds of limiting dilution cell plating. Ibrutinib resistant TMD8 were generated by continuous passaging in a humidified atmosphere of 5% CO₂ and 95% air at 37° C. in the presence of Ibrutinib for 12 weeks then dose-escalating to 10 or 20 nM until resistance to ibrutinib was established. Parallel cultures were grown in the presence of 0.1% v/v DMSO as passage-matched, drug-sensitive control lines. Sensitive and resistant TMD8 cells were clonally isolated through two rounds of single cell limiting dilution. Doubling times and sensitivity to Ibrutinib were evaluated to match the parental line.

Cell Viability Assay:

Resistance to Ibrutinib was determined by comparison of Ibrutinib sensitivity in passage matched DMSO-treated vs. Ibrutinib-treated cultures using a 96-hour CellTiter-Glo viability assay (Promega).

Genotypic Profiling:

Genotypic characterization of Ibrutinib-sensitive and Ibrutinib-resistant clones was evaluated by Sanger hotspot mutational analysis (Genewiz) or by whole exome sequencing (WES) and RNASeq (Expression analysis). DNA sequencing reads were aligned to human reference genome by BWA. Single nucleotide variants were identified using VarScan and were annotated using SnpEff Putative somatic mutations were prioritized by mutant allele frequency, recurrence and predicted functional impact. RNA sequencing reads were aligned to human reference genome using STAR and RNA abundance was quantified using RSEM. The Bioconductor package edgeR was used to normalize sequence count and limma was used to conduct differential gene expression analysis.

Protein Expression and Phosphoproteomics:

Protein expression level and phosphoproteomics were measured using Western Blot or Peggy Sue, as described in Example 3B above.

Results

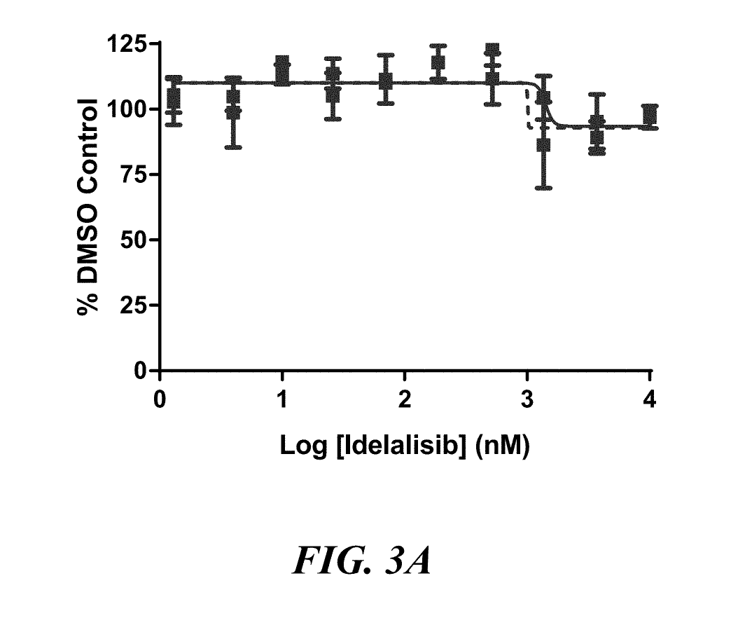

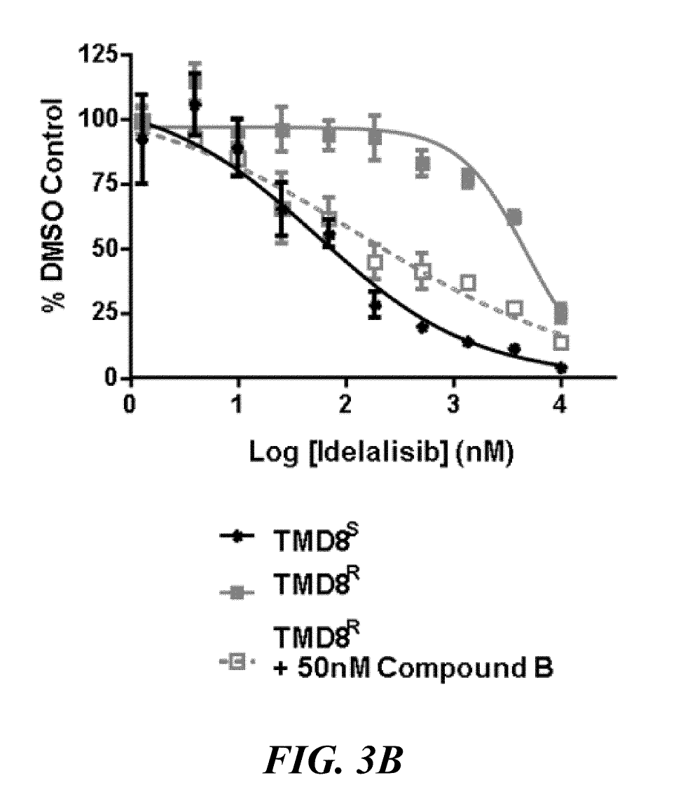

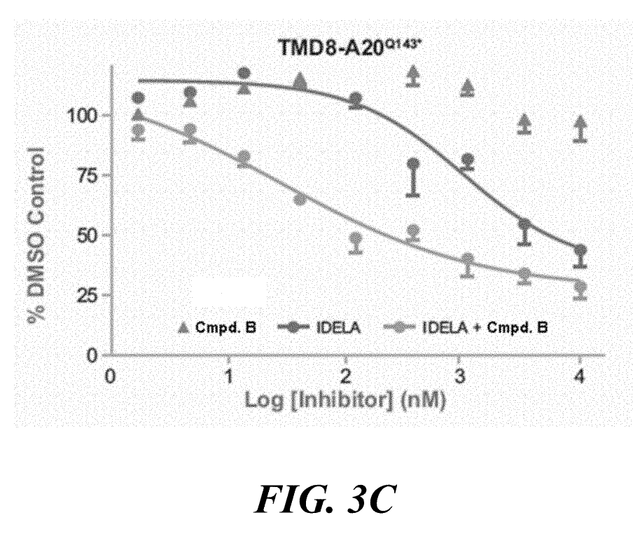

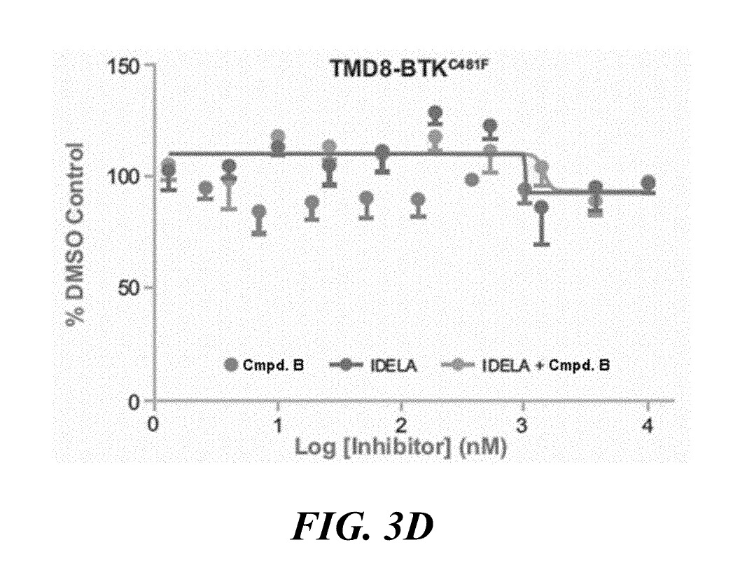

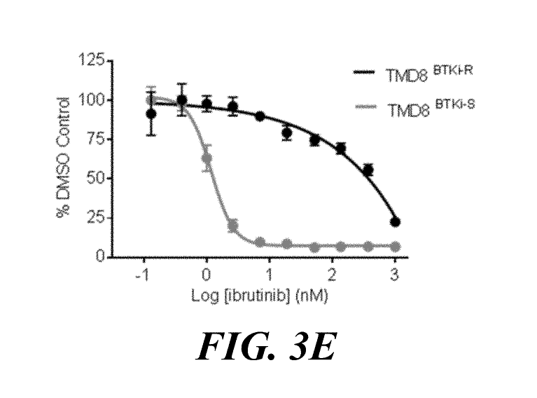

TMD-8 BTK inhibitor-resistant cells were generated by continuous passaging of cells in 10- or 20-nM ibrutinib over several months. A mutation in TNFAIP3 (Q143*, A20 protein) was identified in the 10 nM-treated cells. A mutation in BTK (C481F) was detected in the 20 nM-treated cells, with a concomitant loss of A20 protein. WES analysis of clonal isolates from both lines revealed a homozygous mutation in BTK at C481F only in the 20 nM ibrutinib treated clones (TMD8^{BTK-C481F}, 22/22 clones), and the results were confirmed by Sanger sequencing. WES analysis of the 10 nM ibrutinib treated clones revealed an inactivating mutation in the NF-κB inhibitor TNF alpha induced protein 3 (TNFAIP3 Q143*mutation, also known as A20 protein; TMD8^{A20-Q143*}, 5/5 clones). Cell viability assay in the presence of ibrutinib shows that these clones (TMD8BTKi^{R}) were resistant to ibrutinib (FIG. 3E).

The results of this example are summarized in Table 8 below. The data in Table 8 were generated according to the Western Blot procedure described in Example 3B above.

TABLE 8

| Sample | DMSO | A20^{Q143}* | BTK^{C481F} |
|---|---|---|---|
| A20 | +++ | — | — |
| pIκBα (Ser 32) | 3503 | 4615 | 3448 |
| Total IκBα #4814 | 41508 | 39233 | 41553 |

Protein expression profiling in showed a loss of A20 and an increase in p-IκBα in the TMD8^{A20-Q143*} clone, indicating activation of the NF-κB pathway (Table 8). The TMD8^{BTK-C481F} also showed a loss of A20 by an unknown mechanism. As seen in the table above, the observed acquired mutation of BTK at C481 was in line with ibrutinib clinical resistance, and A20 mutation and loss of function was identified as a mechanism of resistance to a BTK inhibitor.

### Example 4B: Effects of Idelalisib in Combination with Compound B on Resistance to BTK Inhibitors

### Materials and Methods

To evaluate mechanisms of Ibrutinib resistance in TMD-8, several independent clonal isolates of TMD-8 were generated through 2 rounds of limiting dilution cell plating. Doubling times and sensitivity to Ibrutinib were evaluated to match the parental line. Ibrutinib resistance was established by passaging of the clonal isolates in the presence of Ibrutinib in a step-up fashion or, in parallel, in 0.1% v/v DMSO. Resistance to Ibrutinib was ascertained by comparison of Ibrutinib sensitivity in passage matched DMSO-treated vs Ibrutinib-treated cultures using a 96-hour Cell Titer Glo viability assay (Promega). Clonal isolates from Ibrutinib sensitive (DMSO-treated) and Ibrutinib resistant (Ibrutinib treated) were generated through 2 rounds of limiting dilution plating. Genotypic characterization of Ibrutinib-sensitive and Ibrutinib-resistant clones was evaluated by Sanger hotspot mutational analysis (Genewiz) or by whole exome sequencing (WES) (Expression analysis). Sensitivity of Ibrutinib-resistant TMD-8 to PI3K-isoform selective and BTK inhibitors or combinations were performed by treating cells with inhibitors in 10-point dose response for 96 hours followed by performing Cell Titer Glo cell viability assay.

Total protein expression levels and phosphorylation of PI3K, MAPK, BTK and NF-κB components were determined by Western Blot or Peggy Sue. Cells were treated with idelalisib (420 nM), Compound B (320 nM) or in combination. Protein expression level and phosphoproteomics were determined using Western Blot (p-ERK 1/2, p-AKT S473, total AKT) and Peggy Sue (p-BTK, p-IκBα S32, total IκBα), using procedures as described in Example 3B. Results were quantitated after determining the AUC for each group and normalized to DMSO vehicle control.

### Results

Two mechanisms of resistance to BTK inhibitors were identified in TMD-8: an inactivating mutation in the NF-κB inhibitor A20 (TNFAIP3 Q143*), and additionally a BTK mutation (C481F) only present in clones generated with the highest concentration of Ibrutinib (20 nM). TMD-8 cells with the BTK (C481F) mutation only were less sensitive to Idelalisib (Eₘₐₓ=14% at 1 μM vs. 86% in parental, FIG. 3A).

Addition of Compound B did not enhance growth inhibition in those clones. A20 mutant only TMD-8 cells were resistant to Compound B (EC₅₀>10 μM), but were sensitive to Idelalisib, albeit less than parental (EC₅₀≥4300 nM vs. 54 nM). Addition of 50 nM Compound B to Idelalisib provided further growth inhibition, consistent with the presence of wild-type BTK, and increased the potency of Idelalisib to a level comparable to parental TMD-8 (EC₅₀≥99 nM, n=5 clones, FIG. 3B).

The effects of the combination of Idelalisib and Compound B are further illustrated in FIGS. 3C and 3D, and Tables 10 and 11 below. These data show that the combination can overcome BTK-inhibitor resistance in TMD8-A20^{Q143*} by MAPK (mitogen-activated protein kinase) and NF-κB pathway downmodulation. The data in Tables 9 and 10 were generated according to the Western Blot procedure described in Example 3B above. As shown in FIG. 3D, the TMD8^{BTK-C481F} line was resistant to idelalisib, Compound B, and combination thereof, suggesting a complex mechanism of resistance in this line. TMD8^{A20-Q143*} cells were resistant to either idelalisib or Compound B alone, which sensitivity was restored with the combination (FIG. 3C).

Results showed that increased inhibition to p-ERK and p-IκBα in the TMD8^{A20-Q143*} lines was observed in the samples treated the combination of both agents.

TABLE 9
pERK values for TMD-8 Ibrutinib resistant lines

| pERK | | | | Idela + |
|---|---|---|---|---|
| T202/Y204 | DMSO | Idelalisib | Compound B | Compound B |
| control | 1 | 0.13 | 0.35 | 0.24 |
| A20-Q143* | 0.91 | 0.73 | 0.76 | 0.26 |
| BTK-C481F | 1.14 | | | |

TABLE 10
pIκBa values for TMD-8 Ibrutinib resistant lines

| pIκBa | | | | Idela + |
|---|---|---|---|---|
| S32 | DMSO | Idelalisib | Compound B | Compound B |
| control | 1 | 1.09 | 0.62 | 0.55 |
| A20-Q143* | 1.39 | 0.92 | 1.12 | 0.53 |
| BTK-C481F | 0.98 | | | |

Conclusion

A20 mutation and loss-of-function was identified as a novel mechanism of resistance to BTK inhibitors. Idelalisib was observed to less potently inhibit the growth of A20 mutant TMD-8, but the combination with Compound B was observed to provide additional benefit. TMD-8 with a BTK-C481F mutation was resistant to Idelalisib and to the combination with Compound B. These data suggest that the combination of Idelalisib and Compound B may overcome some mechanisms of resistance to BTK. These results suggest that inhibition of MAPK and NF-κB pathways may result in a decreased cell viability observed in this line when treated with a combination of idelalisib and Compound B.

### Example 5: Upregulation of the PI3K Signaling Pathway Mediates Resistance to Idelalisib

In this example, a PI3Kδ-driven model was developed to study mechanism of resistance to Idelalisib. The mechanism of resistance to Idelalisib was also evaluated in a model of ABC-DLBCL (TMD-8). Cell-signaling pathways dysregulated in Idelalisib-resistant cells was also determined. Further, compounds that can overcome Idelalisib resistance were identified.

### Materials and Methods

Growth inhibition to Idelalisib or other inhibitors was assessed using CellTiter-Glo viability assay at 96 h. Idelalisib-resistant line (TMD8R) was generated by continuous exposure to 1 μM idelalisib (˜2× maximum concentration [Cmax] corrected for protein binding); a dimethyl sulfoxide (DMSO) passage matched line was generated as a control (TMD8S). Clonal isolates from pools were generated through 2 rounds of limiting dilution. Cell lines were analyzed by whole exome sequencing, RNASeq, and phosphoproteomics. Protein expression was measured using Simple Western and SDS/PAGE and western blot. Caspase 3/7 was measured using Caspase-Glo 3/7 assay; apoptosis was measured with Annexin V assay and propidium iodide by flow cytometry.

Genomic Profiling:

Gene expression levels and mutations were determined by whole exome sequencing (Genewiz, Inc.) and RNASeq (Expression Analysis), respectively. The following bioinformatics platforms were used to analyze the sequence reads: DNA sequencing reads were aligned to human reference genome by BWA. Single nucleotide variants were identified using VarScan and were annotated using SnpEff Putative somatic mutations were prioritized by mutant allele frequency, recurrence and predicted functional impact. RNA sequencing reads were aligned to human reference genome using STAR and RNA abundance was quantified using RSEM. The Bioconductor package edgeR was used to normalize sequence count and limma was used to conduct differential gene expression analysis.

Western Blot and Protein Expression:

Protein expression was measured using Simple Western, SDS/PAGE and Western Blot or Peggy Sue (ProteinSimple), generally according to the procedures described above in Example 3B. Primary antibodies used to test phosphorylated protein or total protein levels include antibodies against: p-AKT (S473), p-AKT (T308), AKT, p-ERK (T202/Y204), p-S6 (S235/236), S6, p-PDK1 (S241), p-PLCγ2 (Y1217), p-GSK3β (S9), p-STAT3 (Y705), p-IκBα (S32), IκBα, p-SYK (Y525/526), p-BTK (Y223), PI3Kγ, PTEN, and actin.

The compounds used in this example include: (1) Idelalisib (also referred to as “Idela”); (2) monohydrochloride salt of 6-amino-9-[(3R)-1-(2-butynoyl)-3-pyrrolidinyl]-7-(4-phenoxyphenyl)-7,9-dihydro-8H-purin-8-one, referred to in the Examples as Compound B; (3) GDC-0941; (4) BYL-719; (5) AZD-6482; (6) Duvelisib; (7) Ibrutinib; (8) MK-2206; and (9) GSK-2334470.

Statistical Analysis:

Cell viability EC₅₀ was determined using a sigmoidal dose-response (variable slope) curve generated from quadruplicate samples. Statistical significance was determined using the student's t-test for cell viability and two-tailed paired t-test for apoptosis experiments in Prism (GraphPad).

### Results

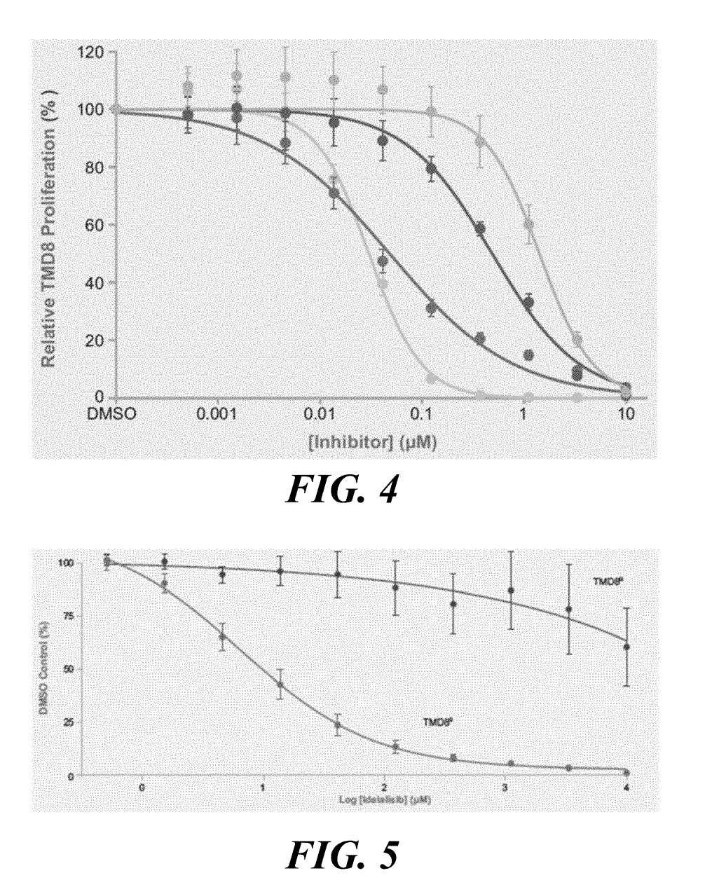

FIG. 4 and Table 11 below show that TMD8 were sensitive to Idelalisib and the pan-PI3K inhibitor (GDC-0941) but not to PI3Kα (BYL-719) or PI3Kβ (AZD-6482) inhibitors, indicating that cell viability is primarily driven by PI3Kδ.

TABLE 11

| | Target PI3K | TMD8 EC₅₀, |
|---|---|---|
| Inhibitor | Isoform | nM |
| Idelalisib | δ | 42 |
| BYL-719 | α | 1200 |
| AZD-6482 | β | 520 |
| GDC-0941 | pan | 27 |

FIG. 5 shows that TMD8 cells with acquired idelalisib resistance (TMD8^{R}) showed a loss of sensitivity to Idelalisib. Growth inhibition was 19% with TMD8^{R} at 1 μM vs 92% with the sensitive DMSO control (TMD8^{S}).

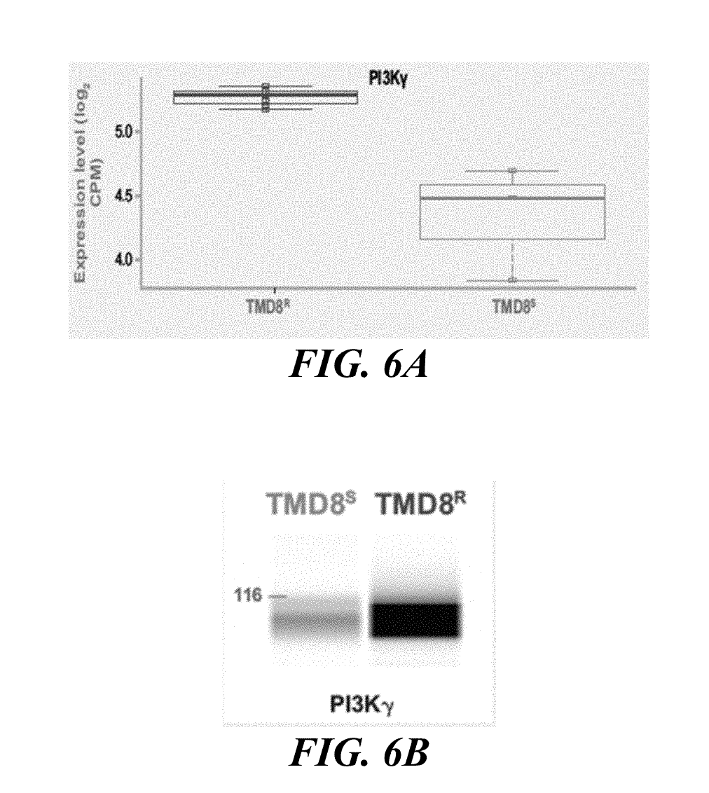

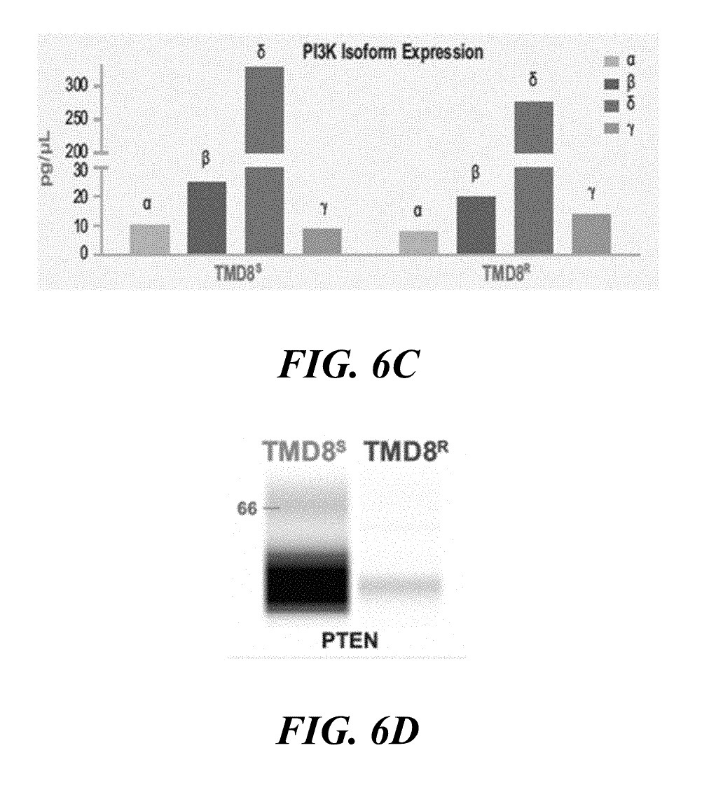

TMD8^{R} profiling shows PI3Kγ upregulation and PTEN loss. FIGS. 6A and 6B show that TMD8^{R} pool and 8/8 clones displayed a modest upregulation of PIK3CG (p110γ) mRNA (2-fold, FIG. 6A) and protein (3-5 fold, FIG. 6B) compared with TMD8^{S}.

FIG. 6C shows that PI3Kδ remained the most prevalent PI3K isoform expressed in TMD8^{R} pool and in 8/8 clones. The levels of PI3Kδ, PI3Kα, PI3Kβ and PI3Kγ were 326.5, 10, 25, and 9 pg/uL, respectively. No mutation in PI3Kδ or other PI3K/AKT pathway members, including PTEN, was found in TMD8^{R} clones by WES. FIG. 6D shows a dramatic reduction (9-fold) of PTEN protein expression was observed.

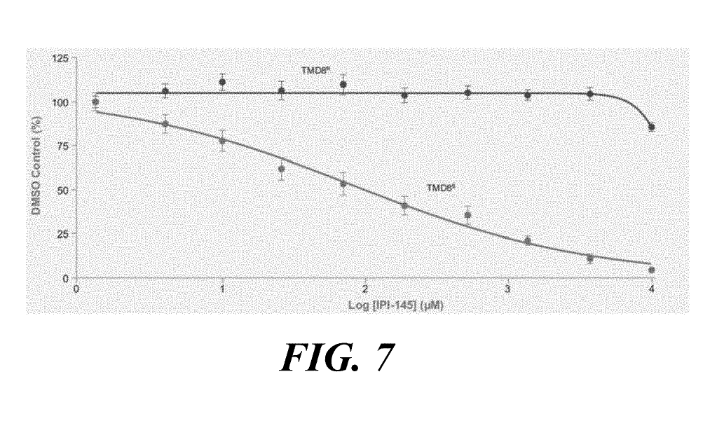

As seen in FIG. 7, TMD8^{R} were observed to be cross-resistant to IPI-145 (Duvelisib), a dual PI3Kδ/γ inhibitor. The EC₅₀ of duvelisib for TMD8^{R} was observed to be >4 μM, whereas the EC₅₀ for TMD8^{S} was observed to be 0.58 μM.

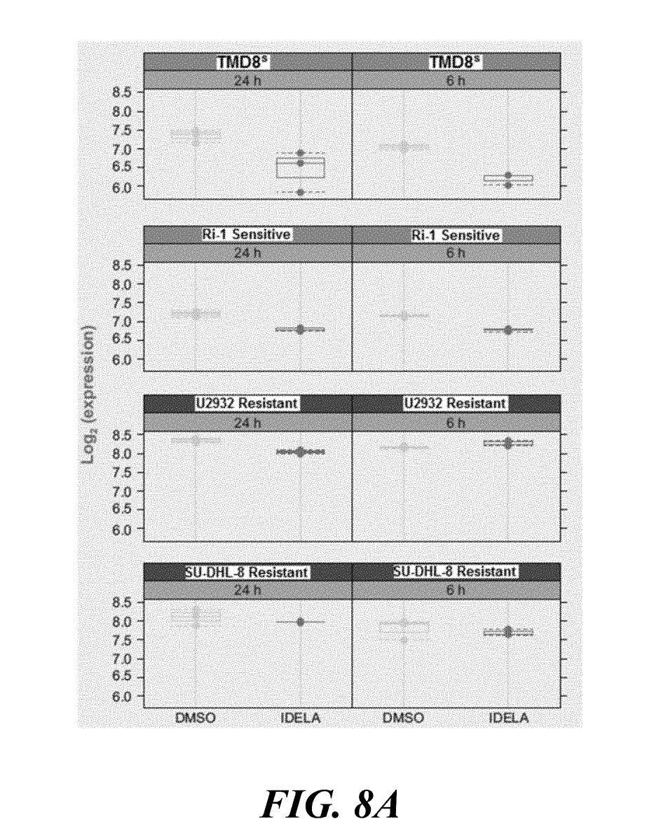

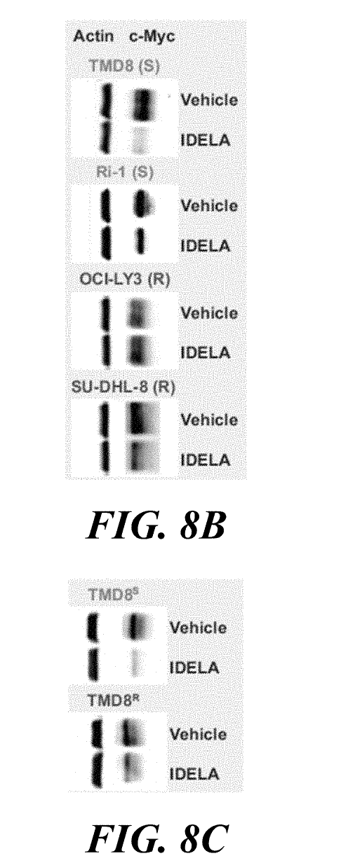

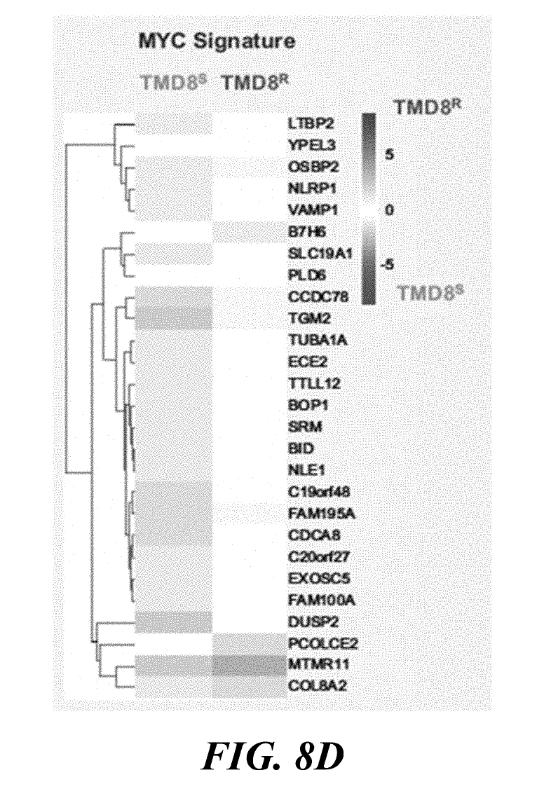

Idelalisib was also observed to downmodulate c-Myc RNA and protein in sensitive but not resistant ABC-DLBCL cell lines. FIG. 8A is a RNAseq analysis of idelalisib-sensitive and -resistant ABC-DLBCL cell lines, which shows that 500 nM idelalisib treatment led to c-Myc mRNA downregulation in sensitive (TMD8 and Ri-1) but not resistant (U2932 and SU-DHL-8) cell lines. In FIG. 8B, RNAseq data were validated by western blot with 500 nM idelalisib for 24 h. As shown in FIG. 8C, c-Myc was inhibited with idelalisib in TMD8^{S} but not TMD8^{R}. In FIG. 8D, expression of c-Myc target genes measured by RNAseq was unchanged in the TMD8^{R} compared with TMD8^{S} cell lines. Loss of c-Myc downregulation by idelalisib was identified as one potential mechanism of resistance. (R), resistant; (S), sensitive.

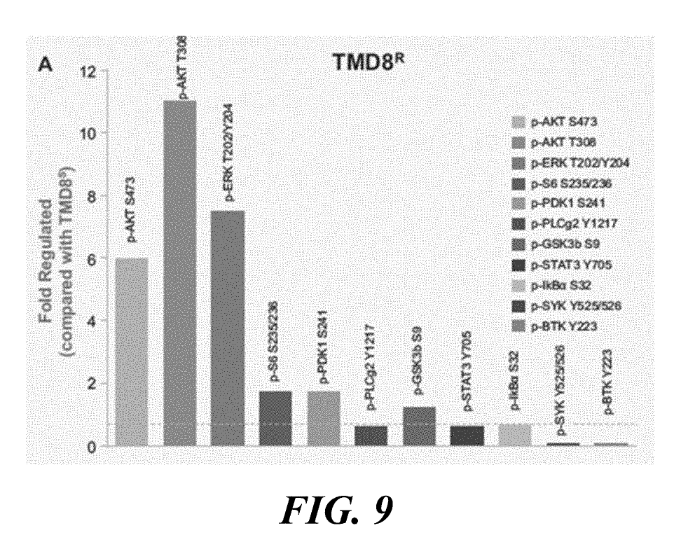

In a phosphoprotein analysis, PI3K and MAPK pathway upregulation in TMD8^{R} was observed with little to no effect on parallel B-cell receptor signaling pathways. The results are shown in FIG. 9 and Table 12. A quantitative analysis of western blots was obtained by densitometry and fold change in TMD8^{S} vs TMD8^{R} was calculated. As seen in FIG. 9, TMD8^{R} showed PI3K and MAPK pathway upregulation in TMD8^{R} while BTK, SYK, JAK, and NF-κB pathways were unchanged. Some pathways were downregulated, as shown by a decrease in p-SYK, p-STAT3 and c-JUN signals. Level of p-ERK and p-SFK remained unchanged. The phosphoproteomic results in Table 12 below for TMD8^{R} were compared with TMD8^{S} cells, and validate the western blot results. The PI3K and MAPK pathway components were upregulated in TMD8^{R} cells, as indicated by the upregulation of p-AKT S473 and T308, p-S6 S235/236, and p-GSK3β, but little to no effect were observed in parallel B-cell receptor signaling pathways.

TABLE 12

| Peptide | Fold Regulated |
|---|---|
| p-AKT3 (472) | 3.8 |
| p-ERK (202, 204) | 2.1 |
| p-PDK (241) | 12 |
| p-S6 (235) | 5.1 |
| S-STAT3 (705) | −1.4 |
| p-IκBα (32, 36) | 0 |
| p-SYK (297) | −1.7 |
| p-BTK (334, 344) | 0 |

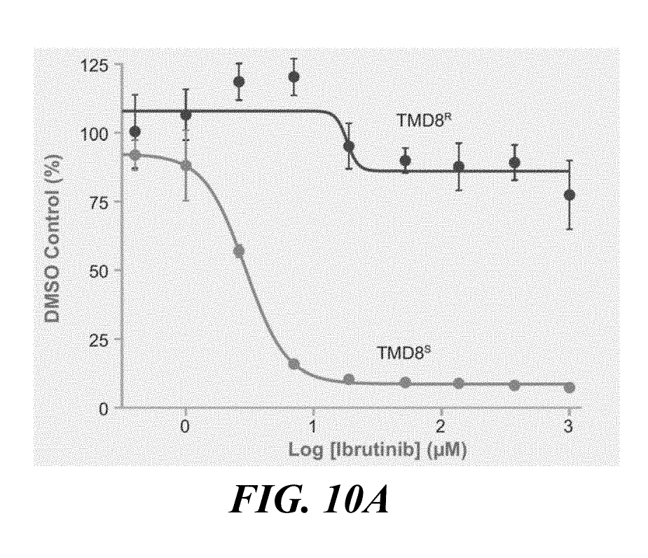

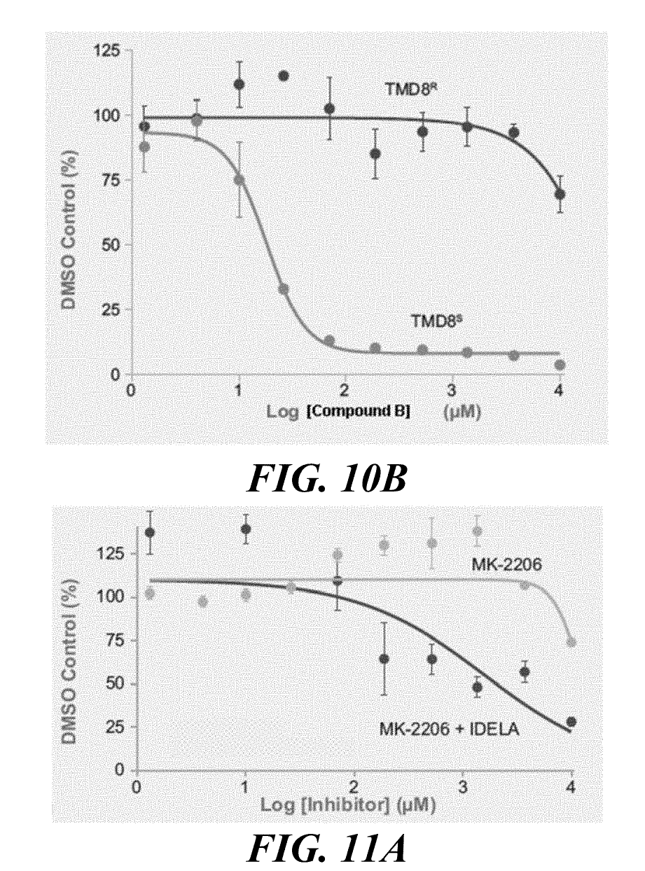

As seen in FIGS. 10A and 10B, TMD8^{R} cells were observed to be cross-resistant to BTK inhibitors, Ibrutinib and Compound B, respectively. The EC₅₀ for Ibrutinib in TMD8^{S} was 0.5, and TMD8^{R} was <10. The EC50 for Compound B in TMD8^{S} was 1.2, and TMD8^{R} was <10.

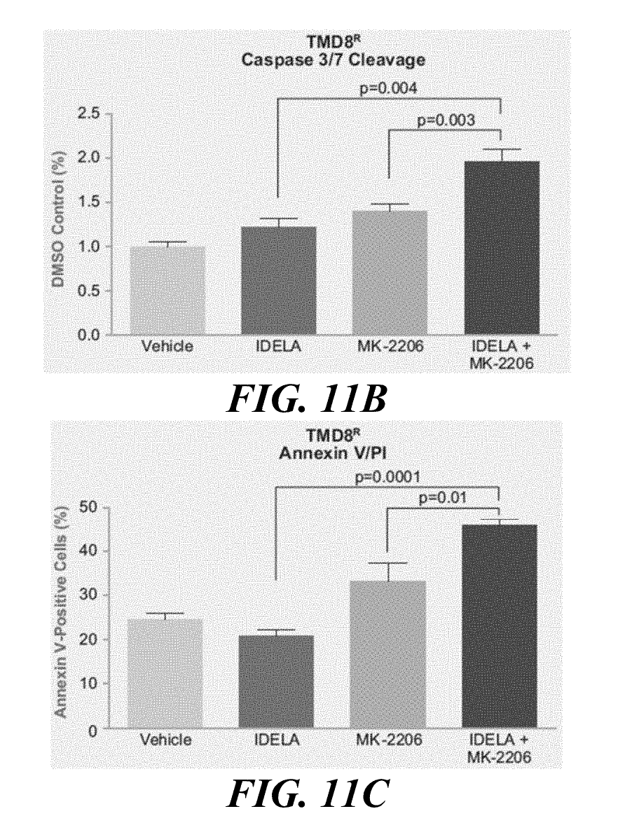

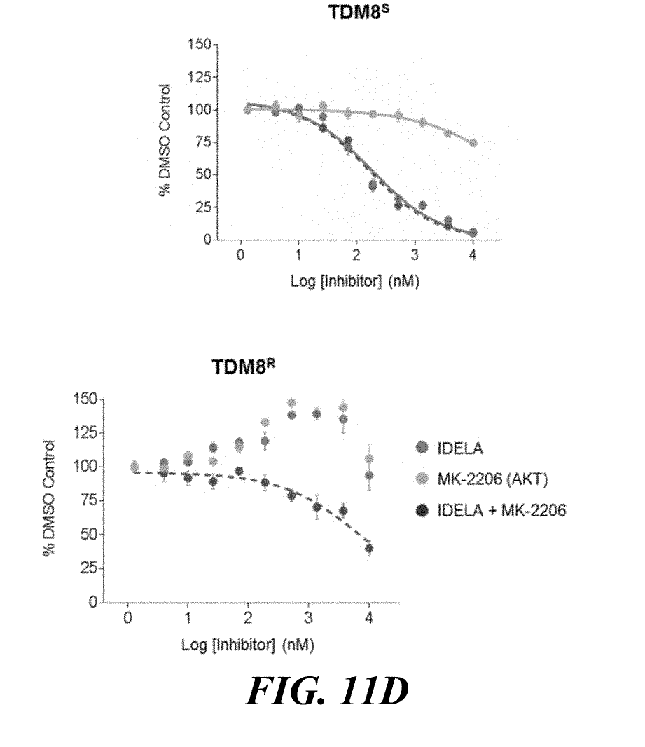

As seen in FIGS. 11A-11C, resistance was observed to be overcome with a combination of MK-2206 (an Akt inhibitor) and Idelalisib. 1 μM MK-2206 EC₅₀<10 μM; 1 μM idelalisib+1 μM MK-2206 EC₅₀=1.6 μM. In FIGS. 11B and 11C, caspase 3/7 was measured at 24 h and Annexin V at 48 h. Idelalisib=1 μM, MK-2206=1 μM; N=4. Two-tailed t-test used to calculate p-values. Increased apoptosis in cells treated with MK-2206 (33%) was observed, compared to vehicle control (24%) and those treated with idelalisib alone (21%). Also, the group treated with a combination of MK-2206 and Idelalisib showed an increase in apoptosis (46%). FIG. 11D shows that the resistance to Idelalisib in TMD8^{R} cells was reduced with a combination of MK-2206 and Idelalisib.

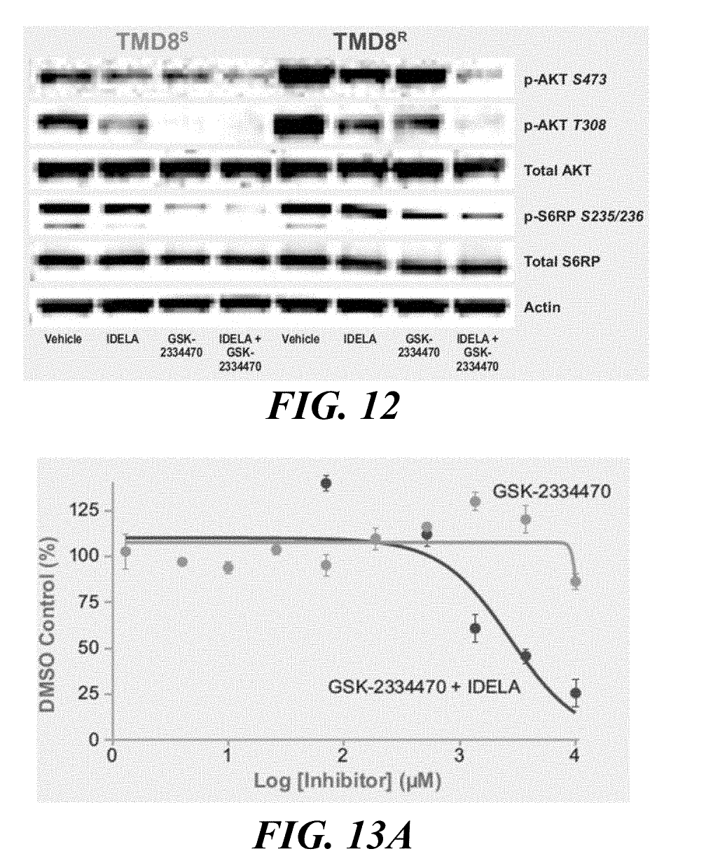

The PI3K pathway inhibition with a combination of MK-2206 and Idelalisib is further illustrated in FIG. 12. In FIG. 12, cells were treated with 1 μM idelalisib, 1 μM MK-2206, or the combination for 2 h. Protein lysates were generated and analyzed by western blot. Increased expression of p-AKT S473, p-AKT T308, and p-S6 S235/236 were seen in TMD8^{R} vs TMD8^{S}; no change was observed in total protein. Greater inhibition of phosphoproteins in TMD8^{S} was observed with single compound compared with TMD8^{R}. The idelalisib and MK-2206 combination resulted in the same inhibition in TMD8^{R} and TMD8^{S} cells. These results suggest that PI3K pathway upregulation in the TMD8^{R} cells may be modulated by combining idelalisib with an AKT inhibitor.

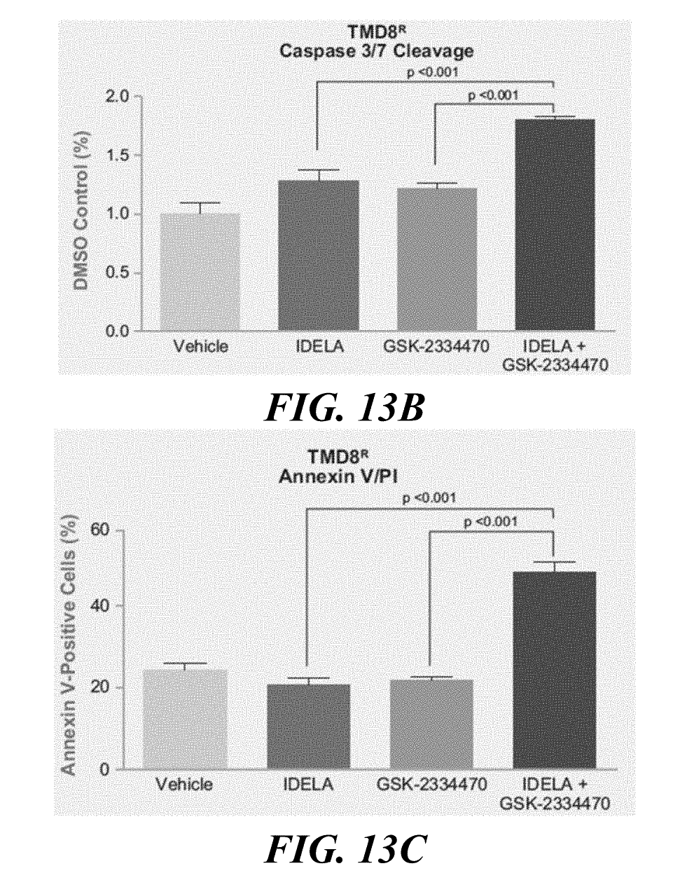

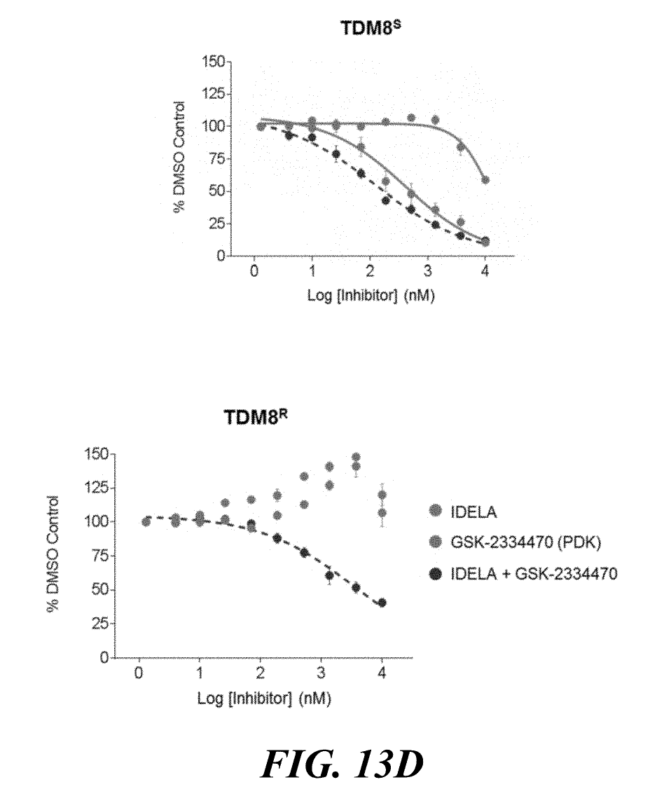

In FIGS. 13A-13C, resistance was observed to be overcome with a combination of GSK-2334470 (a PDK1 inhibitor) and Idelalisib. 1 μM GSK-2334470 EC₅₀<10 μM; 1 μM idelalisib+1 μM GSK-2334470 EC₅₀=1.6 μM. In FIGS. 13B and 13C, caspase 3/7 was measured at 24 h and Annexin V at 48 h. Idelalisib=1 μM, GSK-2334470=1 μM; N=4. Two-tailed t-test used to calculate p-values. PI, propidium iodide. The vehicle control, the cells treated with GSK-2334470 alone or idelalisib alone exhibited apoptosis at 22%, 21%, or 24%, respectively. In comparison, the cells treated with a combination of GSK-2334470 and Idelalisib showed an increase in apoptosis (49%). FIG. 13D shows that resistance to Idelalisib was reduced with a combination of GSK-2334470 and Idelalisib in TMD8^{R} cells.

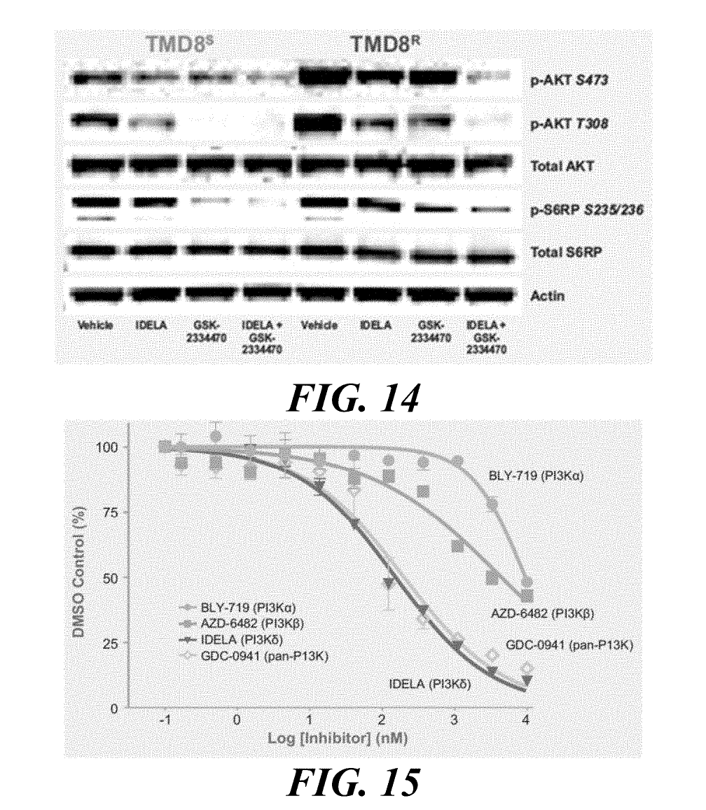

The PI3K pathway inhibition with a combination of GSK-2334470 and Idelalisib is further illustrated in FIG. 14. Cells were treated with vehicle, idelalisib (1 μM), GSK-2334470 (1 μM), or the combination of idelalisib and GSK-2334470 for 2 hours. Protein lysates were analyzed by western blot. Increased basal expression of p-AKT S473, p-AKT T308 and p-S6 S235/236 in TMD8^{R} was observed as compared with TMD8^{S}; no change was observed in total protein. Greater inhibition of phosphoproteins in TMD8^{S} was observed with single compound compared with TMD8^{R}. The combination of idelalisib and GSK-2334470 resulted in the same inhibition in TMD8^{R} and TMD8^{S} cells. These results suggest that PI3K pathway upregulation in the TMD8^{R} cells may be modulated by combining idelalisib with a PDK1 inhibitor.

Thus, the data in this Example shows that treatment with MK-2206 or GSK-2334470 with idelalisib can help to overcome resistance to idelalisib.

### Example 6: Investigation of the Mechanism of Idelalisib Resistance in the Follicular Lymphoma WSU-FSCCL Cell Line

In this example, the mechanisms of resistance to idelalisib in a follicular lymphoma cell line (WSU-FSCCL) were characterized. Further, the effectiveness of other PI3K/protein kinase B (AKT) pathway inhibitors was evaluated to overcome acquired resistance to idelalisib.

### Materials and Methods

Idelalisib resistance was established by continuous passaging of a clonal isolate of WSU-FSCCL in the presence of 1 μM idelalisib; clonal isolates from a passage-matched line (FSCCL^{S}) and idelalisib-resistant line (FSCCL^{R}) were generated through 2 rounds of single-cell-limiting dilution. Growth inhibition to idelalisib or other inhibitors was performed after 96 h using CellTiter Glo viability assay. Characterization of mutations and gene expression were identified by whole exome sequencing and RNA-Seq, respectively. Whole cell lysates were analyzed by western blots.

The compounds used in this example include: (1) Idelalisib (also referred to as “Idela”); (2) GDC-0941; (3) BYL-719; (4) AZD-6482; (5) dasatinib; and (6) entospletinib (also referred to as “Ento”).

### Results

In FIG. 15, FSCCL were observed to be sensitive to PI3Kδ inhibition. FSCCL were observed to be equally sensitive to idelalisib and GDC-0941 (EC₅₀=140 and 180 nM, respectively), and FSCCL were observed to be less sensitive to BYL-719 (EC₅₀>10 μM) and AZD-6482 (EC₅₀=4.6 μM).

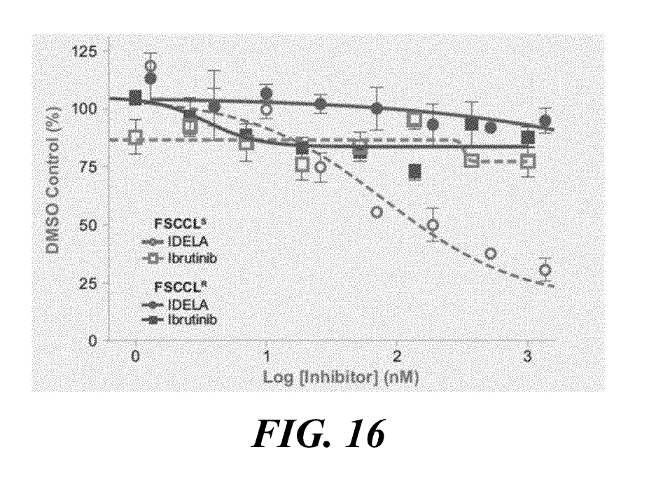

In FIG. 16, FSCCL^{S} and FSCCL^{R} were observed to be less sensitive to ibrutinib (EC50>1 μM), and FSCCL^{S} was observed to be sensitive (EC₅₀=100 nM) and FSCCL^{R} was observed to be less sensitive to idelalisib (EC₅₀>10 μM).

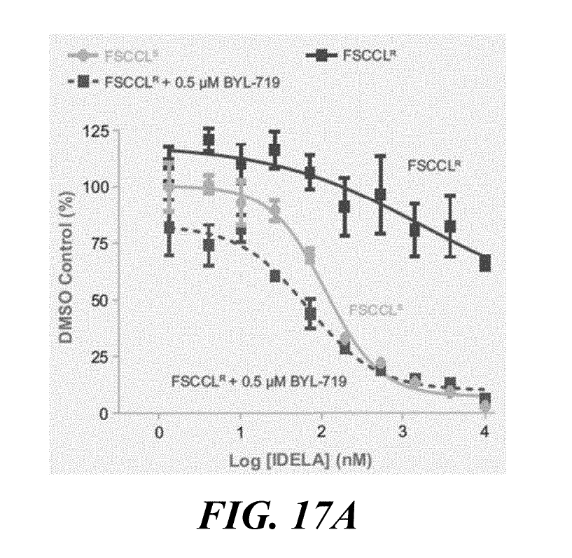

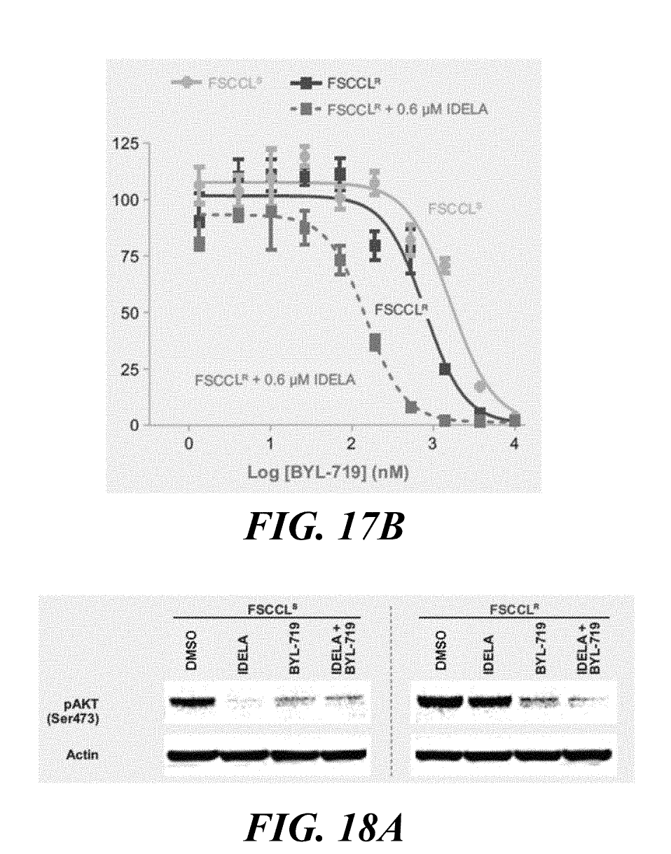

In FIGS. 17A and 17B, FSCCL^{R} PI3KCA mutant (N345K) showed restored sensitivity to the combination of idelalisib and BYL-719. Further, Table 13 below shows viability for the PI3KCA N345K mutant FSCCL^{R} line. Whole exome sequencing analysis revealed PI3KCA resistance mutations in three independently generated sets of FSCCLR clones.

TABLE 13

| | | | | EC₅₀ |
|---|---|---|---|---|
| | | | | BYL-719 |
| | PI3KCA | EC₅₀ | EC₅₀ | (+IDELA |
| | mutation | IDELA, nM | BYL-719, nM | 0.6 μm), nM |
| FSCCL^{S} | WT | 110 | 1613 | NC |
| FSCCL^{R} | E970K | >3000 | 2116 | 123 |
| clone 1 | | | | |
| FSCCL^{R} | N345K | >3000 | 1539 | 143 |
| clone 2 | | | | |
| FSCCL^{R} | P539R | >3000 | >3000 | 239 |
| clone 3 | | | | |

WT = wild-type

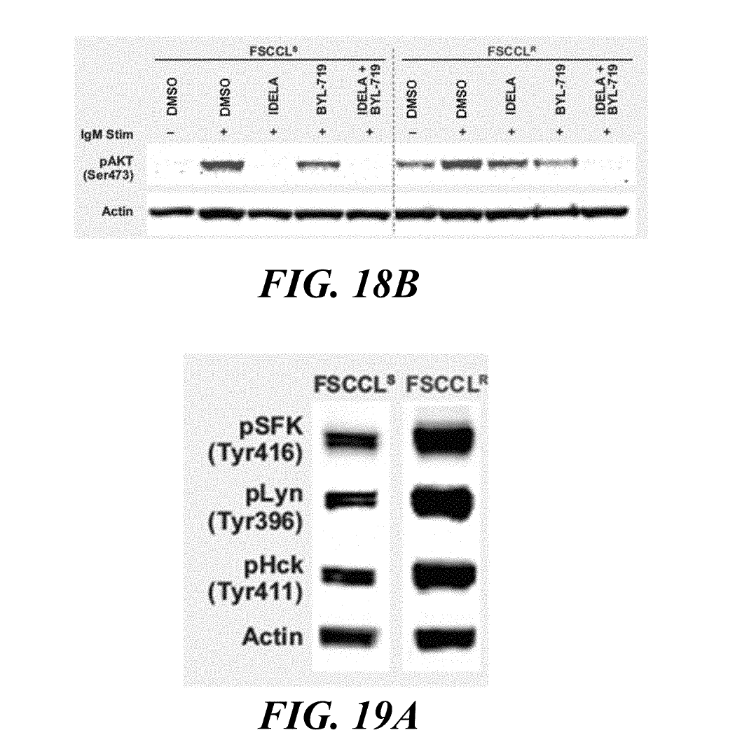

Cells were propagated overnight in drug-free media, then treated for 2 h with either 0.1% DMSO, 600 nM idelalisib, 500 nM BYL-719, or 600 nM idelalisib+500 nM BYL-719. As seen in FIG. 18A, the combination of idelalisib and BYL-719 reduces pAKT (Ser473) expression in FSCCL^{R}. An experiment involving IgM stimulation was also performed. Following overnight growth, cells were starved for 1 h in 0.1% serum before addition of drug (as above). After 2 hours, 5 μg/mL IgM was added to the culture media for 10 min. IgM, immunoglobulin M; pAKT, phosphorylated AKT; Stim, stimulated. As seen in FIG. 18B, the combination of idelalisib and BYL-719 reduces pAKT (Ser473) expression in IgM-stimulated FSCCL^{R}. Thus, while FSCCLR were resistant to idelalisib treatment, the combination of idelalisib and BYL-719 significantly reduced pAKT to levels comparable to the control cell line.

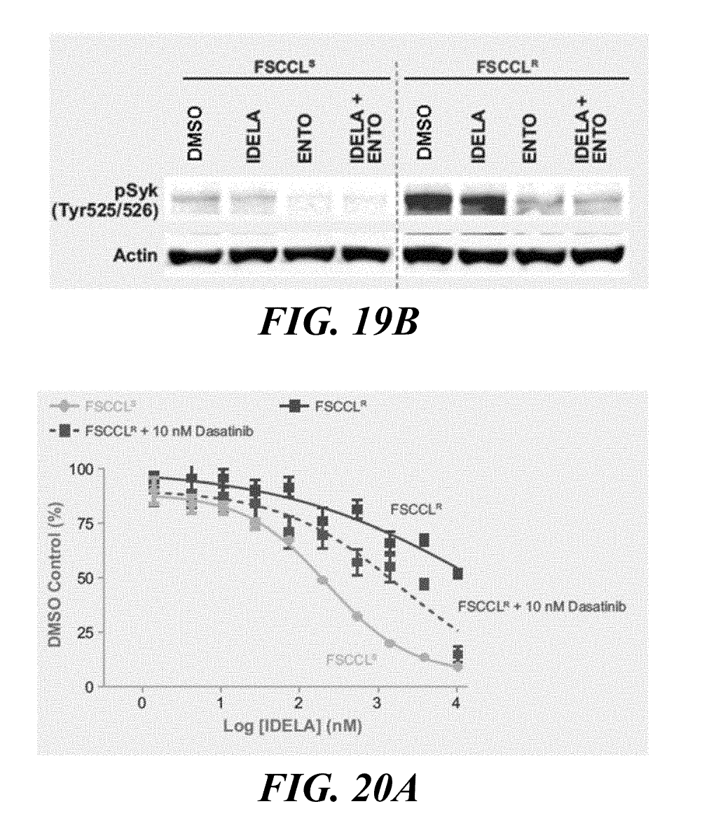

As seen in FIGS. 19A and 19B, FSCCL^{R} SFK^{HIGH} showed an upregulation of SFK phosphorylation (pSFK Tyr416) and phosphorylation of Src family members pHck Tyr411 and pLyn Tyr396 vs FSCCL^{S}.

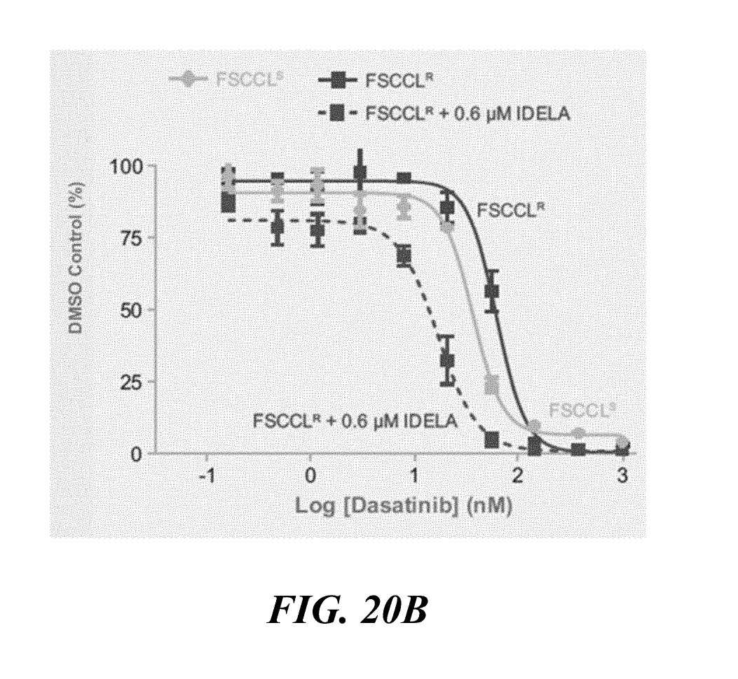

FIGS. 20A and 20B, and Table 14 below, show increased sensitivity of FSCCL^{R} SFK^{HIGH} to the combination of idelalisib and dasatinib.

TABLE 14

| | | EC₅₀ | | EC₅₀ |
|---|---|---|---|---|
| | | IDELA | | Dasatinib |
| | EC₅₀ | (+Dasatinib | EC₅₀ | (+IDELA |
| | IDELA, nM | 10 nM), nM | Dasatinib, nM | 600 nM), nM |
| FSCCL^{S} | 213 | — | 34 | — |
| FSCCL^{R} | — | 1879 | 58 | 15 |

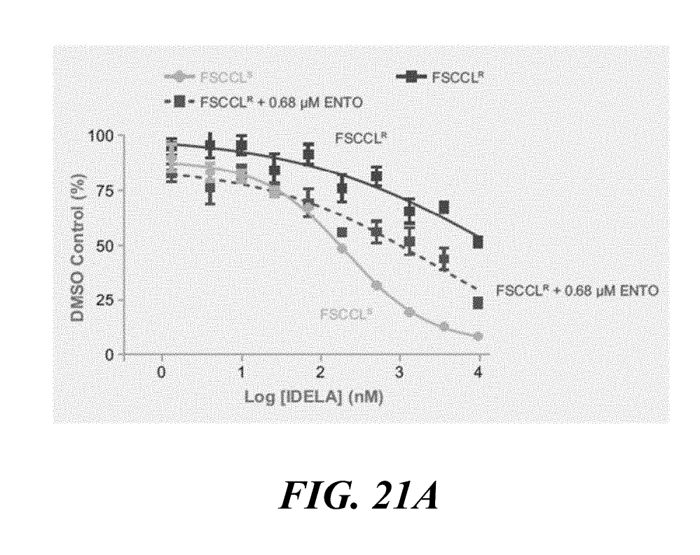

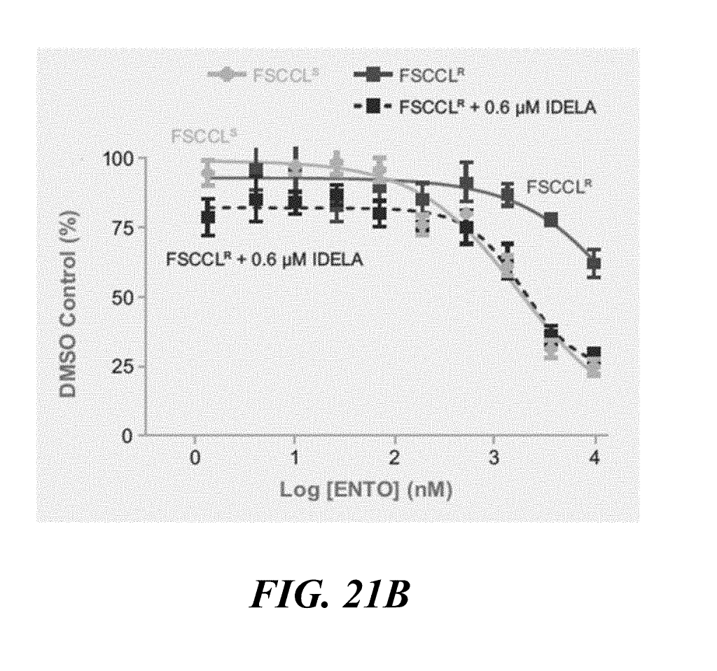

FIGS. 21A and 21B, and Table 15 below, show increased sensitivity of FSCCL^{R} SFK^{HIGH} to the combination of idelalisib and entospletinib, restoring pSyk to FSCCL^{S} levels.

TABLE 15

| | | EC₅₀ | | EC₅₀ |
|---|---|---|---|---|
| | | IDELA | | ENTO |
| | EC₅₀ | (+ENTO | EC₅₀ | (+IDELA |
| | IDELA, nM | 680 nM), nM | ENTO, nM | 600 nM), nM |
| FSCCL^{S} | 213 | — | 1801 | — |
| FSCCL^{R} | — | 2272 | — | 1922 |

Overall, greater sensitivity was observed when a combination of idelalisib and dasatinib, or a combination of idelalisib and entospletinib, was used as compared to the single agents alone.

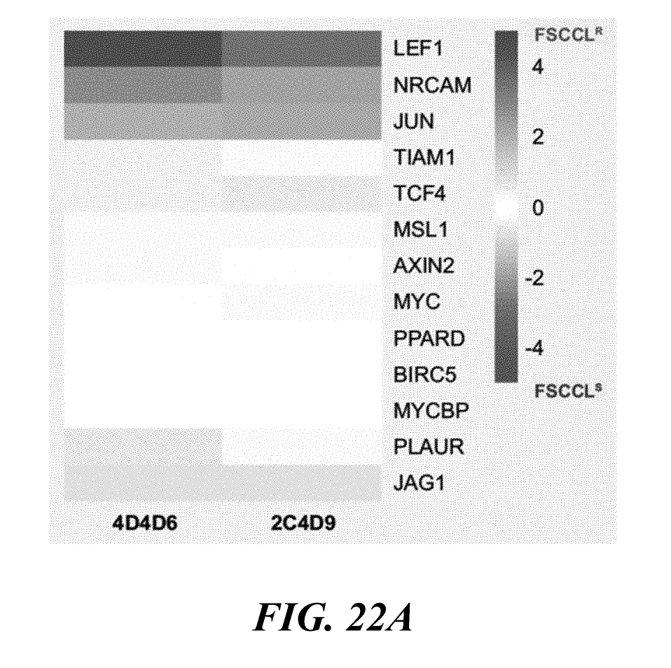

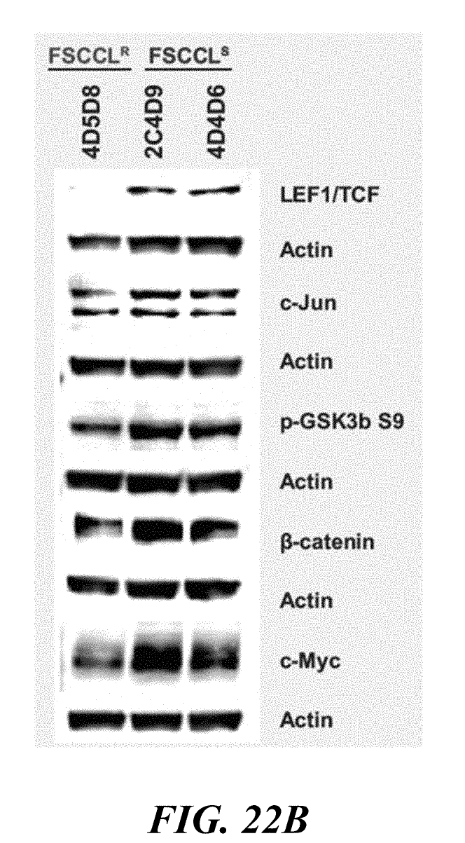

With reference to FIGS. 22A and 22B, a RNA-Seq analysis of the FSCCL^{R}PI3KCA WT single-cell clones revealed that a subset of clones: (1) upregulated Wnt pathway signature, with LEF1 and c-Jun most significantly upregulated in 2 FSCCL^{R} clones; and (2) Western blot analysis confirmed upregulation of LEF1/TCF, c-Jun, β-catenin, c-Myc, and pGSK3β in FSCCL^{R}.

Thus, the data in this example shows that treatment with dasatinib or entospletinib with idelalisib can help to overcome resistance to idelalisib.

### Example 7: Effects of Idelalisib in Combination with Compound B on Resistance to PI3Kδ Inhibitors

### Materials and Methods

Idelalisib-resistant (TMD8^{R}) cell line and passage-matched Idelalisib-sensitive (TMD8^{S}) cell line were generated according to the procedure described in Example 5 above. To characterize the resistant cells, cell viability assays, RNASeq of multidrug resistance (MDR) family of ABC transporter genes (N=33), apoptosis assays, and phosphoprotein analysis were performed. Cell viability was measured using CellTiter-Glo, as described in Example 1A above. Western blot and protein level analysis were performed after treatment with 420 nM idelalisib, 320 nM Compound B and combination thereof, using Peggy Sue (ProteinSimple) automated Western Blot system. Protein concentration (pg/μL) was quantitated using recombinant protein standards. Normalized AUC were determined for each of the treatment groups, normalized to actin. Apoptosis was assessed by propidium iodide and Annexin V/FITC staining and measured by flow cytometry, as described in Example 1A above. Western Blot with anti-p-AKT (S473) antibody and Peggy Sue were used to determine the phosphorylation state of downstream signaling components.

### Results

Cell viability assay showed that the TMD8^{S} cell line remained sensitive to idelalisib, whereas the TMD8^{R} cell lines were resistant to idelalisib treatment (EC₅₀=220 nM, EC₅₀>10 μM, respectively). The acquired resistance was not due to the presence of a subpopulation of innately resistant cells, as the evaluation of 8 single cell clonal isolates all showed resistance to idelalisib (data not shown). Also, the results from RNAseq analysis of the MDR family of ABC transporters in TMD8^{S} and TMD8^{R} cell lines, indicating that upregulation of MDR was not a mechanism of resistance (data not shown).

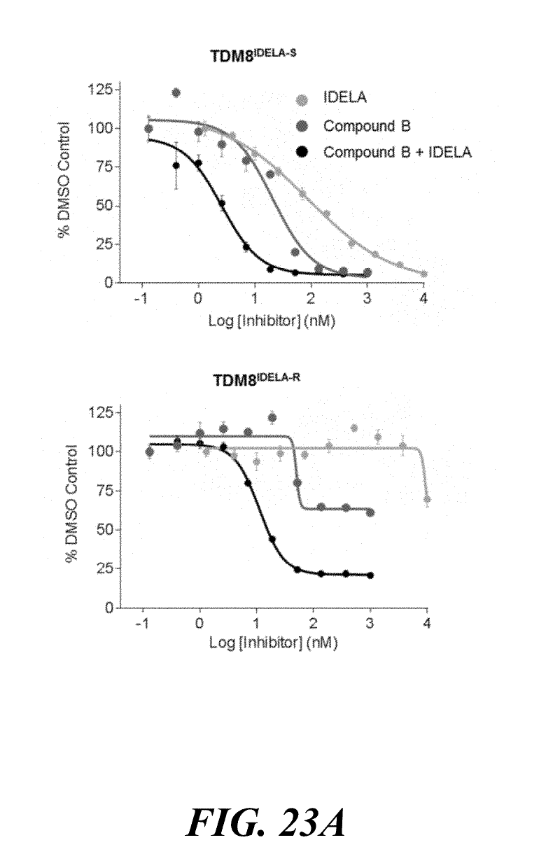

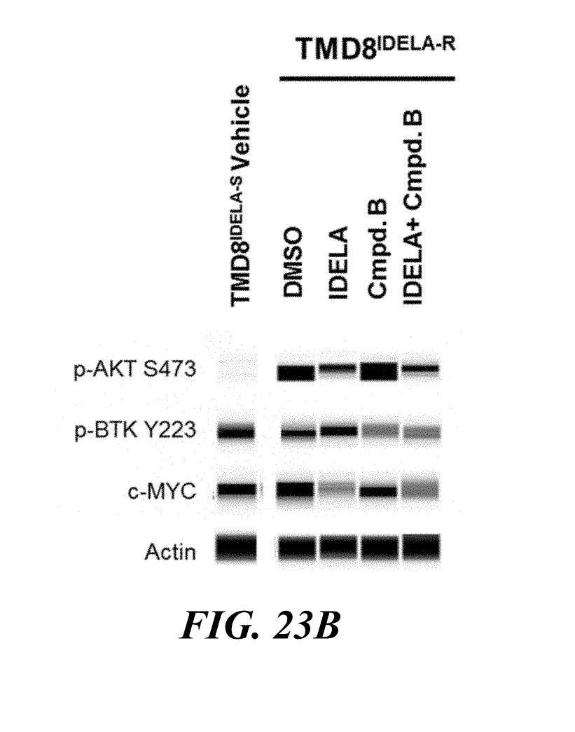

As shown in FIG. 23A, both idelalisib and Compound B inhibited cell growth in the TMD8^{S} cell line but not the TMD8^{R} cell line. Sensitivity of TMD8^{R} cell line was restored when both agents were used in combination.

Also, the results of FIG. 23B shows that Idelalisib alone and combination treatments, but not Compound B treatment, inhibited p-AKT, and that Compound B alone and combination treatments, but not idelalisib treatment, inhibited p-BTK. Also, increased inhibition to cMYC was observed in the cells treated with the combination compared with those of single agent treatment.

### Example 8: Effect of Inhibition of PI3Kδ and BTK on Tumor Regression in Tumor Xenograft Models

Materials and Methods

Tumor Xenograft Model:

A TMD8 tumor xenograft model was generated by introducing cultured TMD8 cells into irradiated mice. All animal experiments were performed in accordance with the Institutional Animal Care and Use Committee (IACUC) protocol. Male CB17-SCID mice were treated with 1.44 Gy whole body irradiation using a ⁶⁰Co radiation source, and seventy-four hours later, 1×10⁷ TMD8 cells were inoculated subcutaneously into the right flank. When tumors reached a mean volume of 200 mm³, mice were randomly assigned to groups (n=13). The groups were administered vehicle, a PI3Kδ inhibitor at 1 and 5 mg/kg or Compound B at 5 and 10 mg/kg, alone or in combination, twice daily, by oral gavage at a dosing volume of 5 mL/kg. All test compounds were formulated in 5% (v/v) N-Methyl-2-pyrrolidone (NMP)/55% (v/v) Polyethylene Glycol 300 (PEG) 300/40% (v/v) Water/1% (w/v) Vitamin E D-α-Tocopherol Polyethylene Glycol 1000 Succinate (TPGS). Tumor volume was calculated using the following formula: (length×width²)/2, where length is the longest diameter across the tumor and width is the corresponding perpendicular diameter. Tumor growth inhibition rate was calculated using the following formula: 1−(tumor size_{end of compound treatment}−tumor size_{beginning of compound treatment}/tumor size_{end of vehicle treatment}−tumor size_{beginning of vehicle treatment})×100.

Western Blots:

Tumor samples obtained from the study were lysed and Western Blot was performed on the samples to determine the level of BTK and S6 phosphorylation, a downstream effector of PI3K signaling. Western Blots were performed according to the Western Blot procedure described in Example 3B above, using antibodies against p-S6 (S235/236), p-BTK (Y223), BTK and actin. Protein levels were determined using Peggy Sue (ProteinSimple) automated Western Blot system. Normalized AUC were determined for each of the treatment groups: p-BTK (Y223) was normalized to total BTK protein, and p-S6 (S235/236) was normalized to actin.

Immunohistochemistry:

Paraffin sections of the tumor samples were prepared for immunohistochemistry. Slides were prepared with EZ Prep (Ventana Medical Systems) CC1 (Ventana Medical Systems) and rinsed with Reaction Buffer (Ventana Medical Systems). Slides were incubated with ChromoMap Inhibitor (Venatana Medical Systems) and rinsed a Reaction Buffer rinse before incubating with anti-pS6 (S235/236) rabbit monoclonal antibody (Cell Signaling Technology) at 0.02 ug/mL or anti-c-MYC rabbit monoclonal antibody (Abcam Inc.) at 0.3 ug/mL. After 1 hour at room temperature, the slides were incubated sequentially with Anti-Rabbit HQ (Ventana Medical Systems), Anti-HQ HRP (Ventana Medical Systems) hydrogen peroxide CM (Ventana Medical Systems), Copper CM (Ventana Medical Systems), and Hematoxylin II. Slides were imaged using the Leica AT2 digital slide scanner (Leica Microsystems Inc.) and documented in Digital Image Hub (DIH-SlidePath).

Statistical Analysis:

The Analysis of Variance model for repeated measure was used to determine the treatment effect on tumor growth. The model fitted on tumor volume included factors of treatment, time, and their interaction. The baseline tumor volume was also included as covariate. The covariance among repeated measurements was assumed with ante-dependence structure. The eight mono and combination treatment groups were compared to vehicle control with Dunnett's multiple comparison adjustment. Each of the four combination treatment groups was also compared to the two corresponding mono dose groups. A multivariate t method was applied for multiple comparison adjustment. A logarithmic transformation was applied on tumor volume to meet model assumptions. The analysis was performed using SAS® 9.2 (SAS Institute, Inc.).

Results

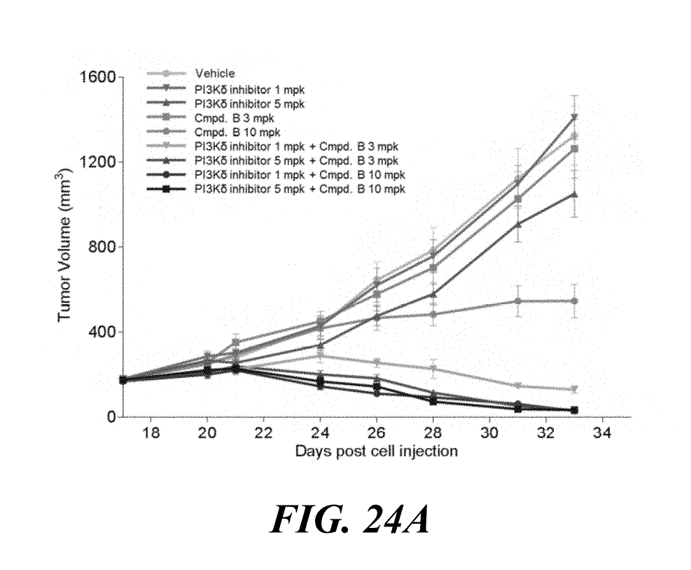

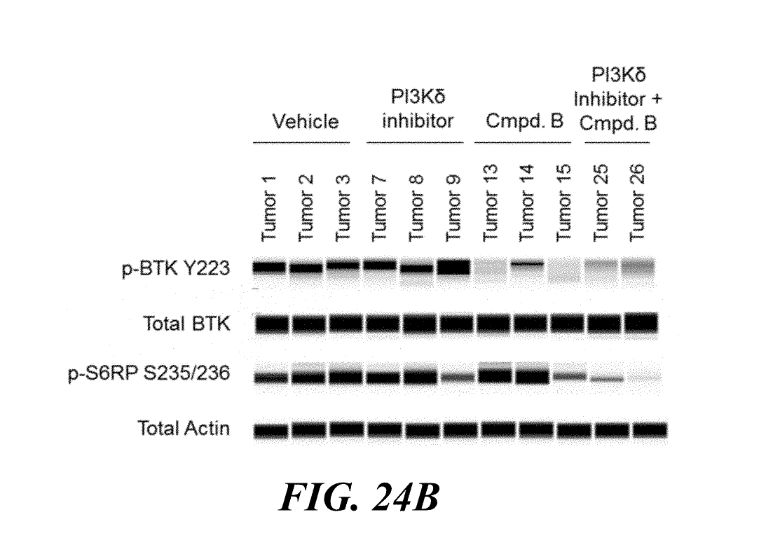

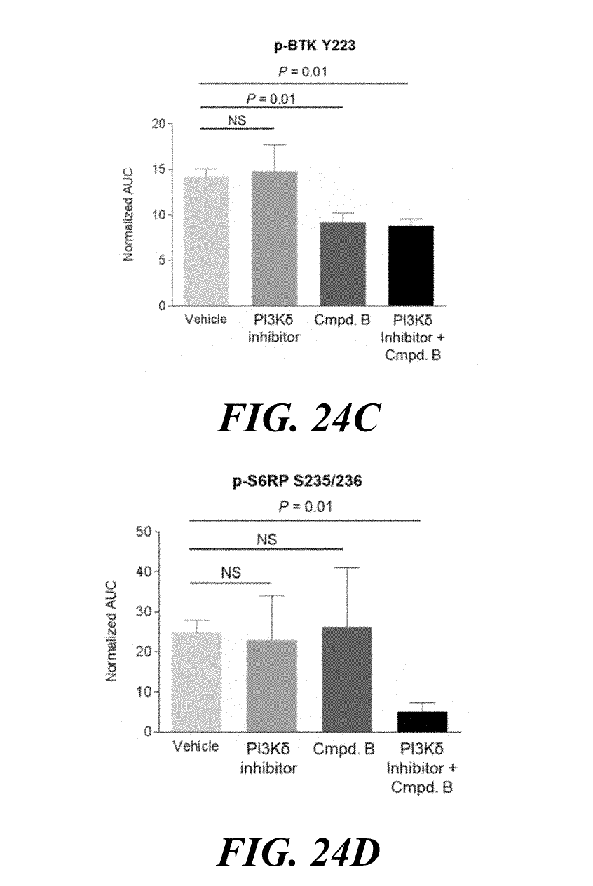

FIG. 24A shows the changes in tumor volume in TDM8 xenograft model mice treated with a combination of a PI3Kδ inhibitor and a BTK inhibitor (Compound B), compared to vehicle control and single agent treatment. Tumor volume assessment showed that the PI3Kδ inhibitor alone did not inhibit tumor growth, at 1 or 5 mg/kg BID, and Compound B singly did not inhibit tumor growth at 3 mg/kg BID, but showed a 75% tumor growth inhibition at 10 mg/kg BID (P<0.05). Mice administered a combination of the PI3Kδ inhibitor and Compound B at both low and high doses exhibited tumor growth inhibition, resulting in tumor regression in all dose combinations tested (P<0.0001).

FIGS. 24B-24D show the results from Western Blot analysis for BTK and PI3K activation in TDM8 xenograft model mice (N=13 per group) treated with a combination of a PI3Kδ inhibitor and a BTK inhibitor (Compound B), compared to vehicle control and single agent treatment. FIGS. 24C and 24D show the quantitation of averages of the tumors for each treatment group (n=3 for vehicle, the PI3Kδ inhibitor and Compound B groups; n=2 for combination). Activation of BTK, as indicated by p-BTK, was reduced by 35% in the Compound B treated group. Compounds B and the PI3Kδ inhibitor each singly did not have an effect on p-S6, but treatment with a combination of Compound B and the PI3Kδ inhibitor exhibited a 79% decrease in p-S6.

Results from the immunohistochemical (IHC) analysis showed reduced p-S6 and c-MYC signal was observed in the group treated with a combination of the PI3Kδ inhibitor (5 mg/kg) and Compound B (10 mg/kg)(data not shown). In comparison, single agent treatment of the PI3Kδ inhibitor (5 mg/kg) or Compound B (10 mg/kg) did not reduce p-S6 S235/236 and c-MYC level (data not shown).

Together, inhibition of both PI3Kδ and BTK signaling pathways showed synergistic effects on multiple signaling pathways, and tumor regression in vivo is observed when inhibitors of both signaling pathways are administered in combination.

1. A method for treating a B-cell malignancy in a human in need thereof, comprising administering to the human
Compound A having the structure 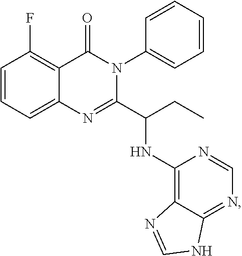 [SMILES: CCC(Nc1ncnc2[nH]cnc12)c1nc2cccc(F)c2c(=O)n1-c1ccccc1]
or a pharmaceutically acceptable salt thereof, at a dose less than or equal to 150 mg; and
a therapeutically effective amount of Compound B having the structure 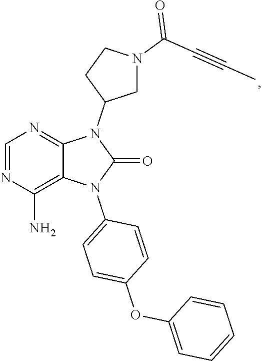 [SMILES: CC#CC(=O)N1CCC(n2c(=O)n(-c3ccc(Oc4ccccc4)cc3)c3c(N)ncnc32)C1]
or a pharmaceutically acceptable salt thereof.

2. The method of claim 1, wherein the dose of Compound A, or a pharmaceutically acceptable salt thereof, is about 50 mg.

3. The method of claim 1, wherein the Compound A, or a pharmaceutically acceptable salt thereof, is administered to the human twice a day.

4. The method of claim 1, wherein the dose of Compound A, or a pharmaceutically acceptable salt thereof, is about 100 mg.

5. The method of claim 1, wherein the Compound A, or a pharmaceutically acceptable salt thereof, is administered to the human once a day.

6. The method of claim 1, wherein the Compound A, or a pharmaceutically acceptable salt thereof is administered orally.

7. The method of claim 1, wherein the Compound B, or a pharmaceutically acceptable salt thereof, is administered to the human at a dose between 1 mg and 200 mg.

10. The method of claim 34, wherein the at least one adverse event is selected from the group consisting of diarrhea, colitis, transaminase elevation, rash, and pneumonitis.

11. The method of claim 34, wherein the administration is at least as effective in inducing anti-proliferative activity in the human as compared to administration of 150 mg of Compound A or Compound B alone to the human.

12. The method of claim 1, wherein the Compound B, or a pharmaceutically acceptable salt thereof, is administered orally.

13. The method of claim 1, wherein the administration of Compound A, or a pharmaceutically acceptable salt thereof, is prior, concurrent or subsequent to the administration of Compound B, or a pharmaceutically acceptable salt thereof.

14. The method of claim 1, wherein:
Compound A, or a pharmaceutically acceptable salt thereof, is present in a pharmaceutical composition comprising Compound A, or a pharmaceutically acceptable salt thereof, and at least one pharmaceutically acceptable excipient; and
Compound B, or a pharmaceutically acceptable salt thereof, is present in a pharmaceutical composition comprising Compound B, or a pharmaceutically acceptable salt thereof, and at least one pharmaceutically acceptable excipient.

15. The method of claim 1, wherein Compound A is Compound A(S) having the structure:

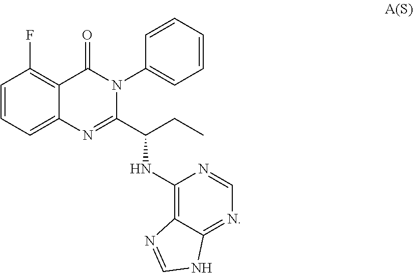 [SMILES: CC[C@H](Nc1ncnc2[nH]cnc12)c1nc2cccc(F)c2c(=O)n1-c1ccccc1]

16. The method of claim 1, wherein Compound B is Compound B(R) having the structure:

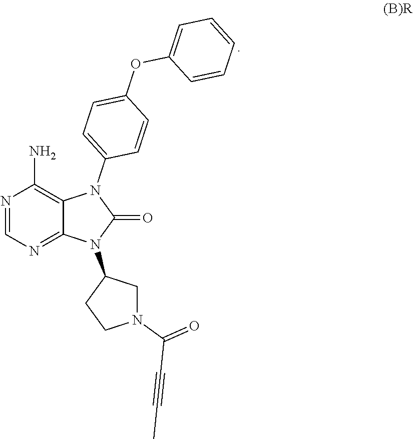 [SMILES: CC#CC(=O)N1CC[C@@H](n2c(=O)n(-c3ccc(Oc4ccccc4)cc3)c3c(N)ncnc32)C1]

17. The method of claim 1, wherein the B-cell malignancy is follicular lymphoma (FL), marginal zone lymphoma (MZL), small lymphocytic lymphoma (SLL), chronic lymphocytic leukemia (CLL), mantle cell lymphoma (MCL), Waldenstrom Macroglobulinemia (WM), non-germinal center B-cell lymphoma (GCB), or diffuse large B-cell lymphoma (DLBCL).

18. (canceled)

19. The method of claim 17, wherein the DLBCL is selected from the group consisting of activated B-cell like diffuse large B-cell lymphoma (ABC-DLBCL) and germinal center B-cell like diffuse large B-cell lymphoma (GCB-DLBCL).

20. (canceled)

21. (canceled)

22. (canceled)

23. (canceled)

24. The method of claim 1, wherein the human who has the B-cell malignancy is (i) refractory to at least one chemotherapy treatment, or (ii) is in relapse after treatment with chemotherapy, or a combination thereof.

25. The method of claim 1, wherein the human has not previously been treated for the B-cell malignancy.

26. A pharmaceutical composition comprising:
Compound A having the structure 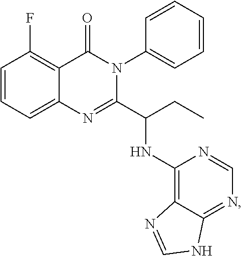 [SMILES: CCC(Nc1ncnc2[nH]cnc12)c1nc2cccc(F)c2c(=O)n1-c1ccccc1]
or a pharmaceutically acceptable salt thereof, at a dose less than or equal to 150 mg; and
a therapeutically effective amount of Compound B having the structure 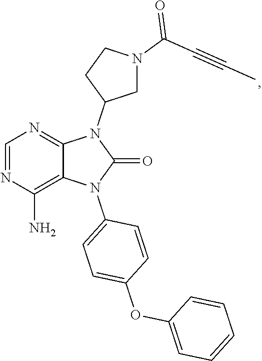 [SMILES: CC#CC(=O)N1CCC(n2c(=O)n(-c3ccc(Oc4ccccc4)cc3)c3c(N)ncnc32)C1]
or a pharmaceutically acceptable salt thereof; and
at least one pharmaceutically acceptable excipient.

27. The pharmaceutical composition of claim 26, wherein the pharmaceutical composition is a tablet.

28. A kit comprising:
a pharmaceutical composition comprising Compound A having the structure 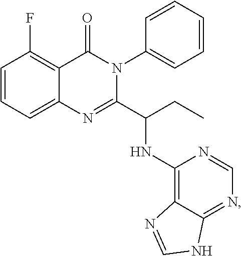 [SMILES: CCC(Nc1ncnc2[nH]cnc12)c1nc2cccc(F)c2c(=O)n1-c1ccccc1]
or a pharmaceutically acceptable salt thereof, present at a dose less than or equal to 150 mg, and at least one pharmaceutically acceptable excipient; and
a pharmaceutical composition comprising Compound B having the structure 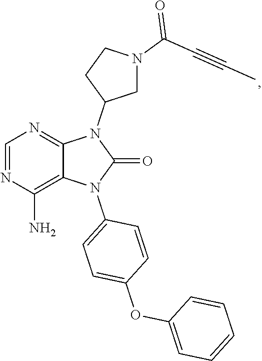 [SMILES: CC#CC(=O)N1CCC(n2c(=O)n(-c3ccc(Oc4ccccc4)cc3)c3c(N)ncnc32)C1]
or a pharmaceutically acceptable salt thereof, and at least one pharmaceutically acceptable excipient.

29. The kit of claim 28, further comprising a package insert containing instructions for use of the pharmaceutical compositions in treating a B-cell malignancy.

30. The kit of claim 28, wherein the B-cell malignancy is selected from the group consisting of follicular lymphoma (FL), marginal zone lymphoma (MZL), small lymphocytic lymphoma (SLL), chronic lymphocytic leukemia (CLL), mantle cell lymphoma (MCL), Waldenstrom Macroglobulinemia (WM), non-germinal center B-cell lymphoma (GCB), or diffuse large B-cell lymphoma (DLBCL).

33. The method of claim 1, wherein the dose of Compound A is between 50 mg and 150 mg.

34. The method of claim 1, wherein the administration reduces or has little to no increase the frequency of at least one adverse event, or the severity of at least one adverse event, or a combination thereof, relative to administration of 150 mg of Compound A alone to the human or relative to administration of the therapeutically effective amount of Compound B alone to the human.
